SEQUENCE LISTING (221 sequences)

SEQ ID NO: 1 - RNA, 20 nt, synthetic construct
  source 1..20: mol_type = other RNA
ttactcgtgtcaaagccgtt

SEQ ID NO: 2 - RNA, 20 nt, synthetic construct
  source 1..20: mol_type = other RNA
gaagcgtgtcttcatagcgc

SEQ ID NO: 3 - RNA, 20 nt, synthetic construct
  source 1..20: mol_type = other RNA
ctttagccactccgcaagcc

SEQ ID NO: 4 - RNA, 20 nt, synthetic construct
  source 1..20: mol_type = other RNA
cctgaaaaggtgtacggcag

SEQ ID NO: 5 - RNA, 20 nt, synthetic construct
  source 1..20: mol_type = other RNA
ccactgccgtacaccttttc

SEQ ID NO: 6 - RNA, 20 nt, synthetic construct
  source 1..20: mol_type = other RNA
tgtcaaagccgttaggatcc

SEQ ID NO: 7 - RNA, 20 nt, synthetic construct
  source 1..20: mol_type = other RNA
tgggccgcttacctgttggc

SEQ ID NO: 8 - RNA, 20 nt, synthetic construct
  source 1..20: mol_type = other RNA
gaattgggccgcttacctgt

SEQ ID NO: 9 - RNA, 20 nt, synthetic construct
  source 1..20: mol_type = other RNA
cactgccgtacaccttttca

SEQ ID NO: 10 - RNA, 20 nt, synthetic construct
  source 1..20: mol_type = other RNA
taggatcctggcttgcggag

SEQ ID NO: 11 - RNA, 20 nt, synthetic construct
  source 1..20: mol_type = other RNA
ggtgtacggcagtggcgaga

SEQ ID NO: 12 - RNA, 20 nt, synthetic construct
  source 1..20: mol_type = other RNA
gcggagtggctaaagtgctt

SEQ ID NO: 13 - RNA, 20 nt, synthetic construct
  source 1..20: mol_type = other RNA
ccgaagtctgtttgcactgc

SEQ ID NO: 14 - RNA, 20 nt, synthetic construct
  source 1..20: mol_type = other RNA
ggctggccgggtgatagtgg

SEQ ID NO: 15 - RNA, 20 nt, synthetic construct
  source 1..20: mol_type = other RNA
aacgaccctgaaaaggtgta

SEQ ID NO: 16 - RNA, 20 nt, synthetic construct
  source 1..20: mol_type = other RNA
ggtggctggccgggtgatag

SEQ ID NO: 17 - RNA, 20 nt, synthetic construct
  source 1..20: mol_type = other RNA
tccgcaagccaggatcctaa

SEQ ID NO: 18 - RNA, 20 nt, synthetic construct
  source 1..20: mol_type = other RNA
cgaagtctgtttgcactgct

SEQ ID NO: 19 - RNA, 20 nt, synthetic construct
  source 1..20: mol_type = other RNA
tcaccatgatggaactgagt

SEQ ID NO: 20 - DNA, 20 bp, synthetic construct
  source 1..20: mol_type = other DNA
gccgttaggatcctggcttg

SEQ ID NO: 21 - RNA, 20 nt, synthetic construct
  source 1..20: mol_type = other RNA
ggtctttaacgaccctgaaa

SEQ ID NO: 22 - RNA, 20 nt, synthetic construct
  source 1..20: mol_type = other RNA
ctatgaagacacgcttcttc

SEQ ID NO: 23 - RNA, 20 nt, synthetic construct
  source 1..20: mol_type = other RNA
cttctggaagaccagccaac

SEQ ID NO: 24 - RNA, 20 nt, synthetic construct
  source 1..20: mol_type = other RNA
gtacggcagtggcgagaagg

SEQ ID NO: 25 - RNA, 20 nt, synthetic construct
  source 1..20: mol_type = other RNA
cacacacctccactatcacc

SEQ ID NO: 26 - RNA, 20 nt, synthetic construct
  source 1..20: mol_type = other RNA
ggctaaagtgctttggatgc

SEQ ID NO: 27 - RNA, 20 nt, synthetic construct
  source 1..20: mol_type = other RNA
aaaccaactcagttccatca

SEQ ID NO: 28 - RNA, 20 nt, synthetic construct
  source 1..20: mol_type = other RNA
gctaaagtgctttggatgca

SEQ ID NO: 29 - RNA, 20 nt, synthetic construct
  source 1..20: mol_type = other RNA
cttcttgaacatcaccatga

SEQ ID NO: 30 - RNA, 20 nt, synthetic construct
  source 1..20: mol_type = other RNA
aagaccagccaacaggtaag

SEQ ID NO: 31 - RNA, 20 nt, synthetic construct
  source 1..20: mol_type = other RNA
gaagatcaagtcttttgagg

SEQ ID NO: 32 - RNA, 20 nt, synthetic construct
  source 1..20: mol_type = other RNA
ccagcagtgcaaacagactt

SEQ ID NO: 33 - RNA, 20 nt, synthetic construct
  source 1..20: mol_type = other RNA
caagaagatcaagtcttttg

SEQ ID NO: 34 - RNA, 20 nt, synthetic construct
  source 1..20: mol_type = other RNA
ggcagtggcgagaaggtggc

SEQ ID NO: 35 - RNA, 20 nt, synthetic construct
  source 1..20: mol_type = other RNA
gtggcgagaaggtggctggc

SEQ ID NO: 36 - RNA, 20 nt, synthetic construct
  source 1..20: mol_type = other RNA
tggcgagaaggtggctggcc

SEQ ID NO: 37 - RNA, 20 nt, synthetic construct
  source 1..20: mol_type = other RNA
gttcggctttgagcttcctc

SEQ ID NO: 38 - RNA, 20 nt, synthetic construct
  source 1..20: mol_type = other RNA
ttagctgatatttacccctg

SEQ ID NO: 39 - RNA, 20 nt, synthetic construct
  source 1..20: mol_type = other RNA
gagatggtgatcatgagacc

SEQ ID NO: 40 - RNA, 20 nt, synthetic construct
  source 1..20: mol_type = other RNA
ccatctcattctcacctgaa

SEQ ID NO: 41 - RNA, 20 nt, synthetic construct
  source 1..20: mol_type = other RNA
ttcggctttgagcttcctca

SEQ ID NO: 42 - RNA, 20 nt, synthetic construct
  source 1..20: mol_type = other RNA
aacaaatatgagtacaagtt

SEQ ID NO: 43 - RNA, 20 nt, synthetic construct
  source 1..20: mol_type = other RNA
accatctcattctcacctga

SEQ ID NO: 44 - RNA, 20 nt, synthetic construct
  source 1..20: mol_type = other RNA
tcggctttgagcttcctcag

SEQ ID NO: 45 - RNA, 20 nt, synthetic construct
  source 1..20: mol_type = other RNA
cccttcaggtgagaatgaga

SEQ ID NO: 46 - RNA, 20 nt, synthetic construct
  source 1..20: mol_type = other RNA
ttgtactcatatttgtttcc

SEQ ID NO: 47 - RNA, 20 nt, synthetic construct
  source 1..20: mol_type = other RNA
aatatgggtgtgtagactac

SEQ ID NO: 48 - RNA, 20 nt, synthetic construct
  source 1..20: mol_type = other RNA
atatgggtgtgtagactact

SEQ ID NO: 49 - RNA, 20 nt, synthetic construct
  source 1..20: mol_type = other RNA
atggatctcaccattaaatc

SEQ ID NO: 50 - RNA, 20 nt, synthetic construct
  source 1..20: mol_type = other RNA
cttgagttggctggctcggg

SEQ ID NO: 51 - RNA, 20 nt, synthetic construct
  source 1..20: mol_type = other RNA
aatatgggtgtgtagactac

SEQ ID NO: 52 - RNA, 20 nt, synthetic construct
  source 1..20: mol_type = other RNA
gtgtgtagactactgggtga

SEQ ID NO: 53 - RNA, 20 nt, synthetic construct
  source 1..20: mol_type = other RNA
atatgggtgtgtagactact

SEQ ID NO: 54 - RNA, 20 nt, synthetic construct
  source 1..20: mol_type = other RNA
aaactttgaagtagtggatc

SEQ ID NO: 55 - RNA, 20 nt, synthetic construct
  source 1..20: mol_type = other RNA
gtctcttgagttggctggct

SEQ ID NO: 56 - RNA, 20 nt, synthetic construct
  source 1..20: mol_type = other RNA
cctctgggaacatccttcaa

SEQ ID NO: 57 - RNA, 20 nt, synthetic construct
  source 1..20: mol_type = other RNA
ggatctcaccattaaatcag

SEQ ID NO: 58 - RNA, 20 nt, synthetic construct
  source 1..20: mol_type = other RNA
ctttgaagtagtggatctgg

SEQ ID NO: 59 - RNA, 20 nt, synthetic construct
  source 1..20: mol_type = other RNA
tgtcaatacccctgatttaa

SEQ ID NO: 60 - RNA, 20 nt, synthetic construct
  source 1..20: mol_type = other RNA
tctcttgagttggctggctc

SEQ ID NO: 61 - RNA, 20 nt, synthetic construct
  source 1..20: mol_type = other RNA
tctttgtctcttgagttggc

SEQ ID NO: 62 - RNA, 20 nt, synthetic construct
  source 1..20: mol_type = other RNA
tggatctcaccattaaatca

SEQ ID NO: 63 - RNA, 20 nt, synthetic construct
  source 1..20: mol_type = other RNA
attttcttgacacaggcctc

SEQ ID NO: 64 - RNA, 20 nt, synthetic construct
  source 1..20: mol_type = other RNA
ttttcttgacacaggcctct

SEQ ID NO: 65 - RNA, 20 nt, synthetic construct
  source 1..20: mol_type = other RNA
cctttgaaggatgttcccag

SEQ ID NO: 66 - RNA, 20 nt, synthetic construct
  source 1..20: mol_type = other RNA
gtgtgtagactactgggtga

SEQ ID NO: 67 - RNA, 20 nt, synthetic construct
  source 1..20: mol_type = other RNA
aattttcattttcttgacac

SEQ ID NO: 68 - RNA, 20 nt, synthetic construct
  source 1..20: mol_type = other RNA
ctttgaagtagtggatctgg

SEQ ID NO: 69 - RNA, 20 nt, synthetic construct
  source 1..20: mol_type = other RNA
catccttcaaaggaaaatat

SEQ ID NO: 70 - RNA, 20 nt, synthetic construct
  source 1..20: mol_type = other RNA
acatccttcaaaggaaaata

SEQ ID NO: 71 - RNA, 20 nt, synthetic construct
  source 1..20: mol_type = other RNA
acacccatattttcctttga

SEQ ID NO: 72 - RNA, 20 nt, synthetic construct
  source 1..20: mol_type = other RNA
tttttctttgtctcttgagt

SEQ ID NO: 73 - RNA, 20 nt, synthetic construct
  source 1..20: mol_type = other RNA
aaagaaaaactttgaagtag

SEQ ID NO: 74 - RNA, 20 nt, synthetic construct
  source 1..20: mol_type = other RNA
gagacagacacccgcccatc

SEQ ID NO: 75 - RNA, 20 nt, synthetic construct
  source 1..20: mol_type = other RNA
cacaggtgcctatatagaag

SEQ ID NO: 76 - RNA, 20 nt, synthetic construct
  source 1..20: mol_type = other RNA
gacacaggtgcctatataga

SEQ ID NO: 77 - RNA, 20 nt, synthetic construct
  source 1..20: mol_type = other RNA
acacaggtgcctatatagaa

SEQ ID NO: 78 - RNA, 20 nt, synthetic construct
  source 1..20: mol_type = other RNA
cccatcaggaatgaacatgc

SEQ ID NO: 79 - RNA, 20 nt, synthetic construct
  source 1..20: mol_type = other RNA
tctgctcgaattgacagaaa

SEQ ID NO: 80 - RNA, 20 nt, synthetic construct
  source 1..20: mol_type = other RNA
aggtaaaatcctagtgctta

SEQ ID NO: 81 - RNA, 20 nt, synthetic construct
  source 1..20: mol_type = other RNA
gcatgttcattcctgatggg

SEQ ID NO: 82 - RNA, 20 nt, synthetic construct
  source 1..20: mol_type = other RNA
catgttcattcctgatgggc

SEQ ID NO: 83 - RNA, 20 nt, synthetic construct
  source 1..20: mol_type = other RNA
cctgcatgttcattcctgat

SEQ ID NO: 84 - RNA, 20 nt, synthetic construct
  source 1..20: mol_type = other RNA
tcctgcatgttcattcctga

SEQ ID NO: 85 - RNA, 20 nt, synthetic construct
  source 1..20: mol_type = other RNA
ttttcttttttagcagacac

SEQ ID NO: 86 - RNA, 20 nt, synthetic construct
  source 1..20: mol_type = other RNA
gacagaaaaggattctgtga

SEQ ID NO: 87 - RNA, 20 nt, synthetic construct
  source 1..20: mol_type = other RNA
gtaagtgtggcgggccacaa

SEQ ID NO: 88 - RNA, 20 nt, synthetic construct
  source 1..20: mol_type = other RNA
gaatacatgttcccgaattg

SEQ ID NO: 89 - RNA, 20 nt, synthetic construct
  source 1..20: mol_type = other RNA
gcctgatcttctgaacccga

SEQ ID NO: 90 - RNA, 20 nt, synthetic construct
  source 1..20: mol_type = other RNA
gtaagtgtggcgggccacaa

SEQ ID NO: 91 - RNA, 20 nt, synthetic construct
  source 1..20: mol_type = other RNA
gggacatgcgcatcatggcg

SEQ ID NO: 92 - RNA, 20 nt, synthetic construct
  source 1..20: mol_type = other RNA
gtaaggaatattcaactcga

SEQ ID NO: 93 - RNA, 20 nt, synthetic construct
  source 1..20: mol_type = other RNA
ccttactggtgggtagatgc

SEQ ID NO: 94 - RNA, 20 nt, synthetic construct
  source 1..20: mol_type = other RNA
tctcagggacatgcgcatca

SEQ ID NO: 95 - RNA, 20 nt, synthetic construct
  source 1..20: mol_type = other RNA
cgccacacttaccttgccaa

SEQ ID NO: 96 - RNA, 20 nt, synthetic construct
  source 1..20: mol_type = other RNA
gccttcgggttcagaagatc

SEQ ID NO: 97 - RNA, 20 nt, synthetic construct
  source 1..20: mol_type = other RNA
gaagatcaggccttctatcc

SEQ ID NO: 98 - RNA, 20 nt, synthetic construct
  source 1..20: mol_type = other RNA
catggcgtggcaagagcctt

SEQ ID NO: 99 - RNA, 20 nt, synthetic construct
  source 1..20: mol_type = other RNA
tcgagttgaatattccttac

SEQ ID NO: 100 - RNA, 20 nt, synthetic construct
  source 1..20: mol_type = other RNA
atggcgtggcaagagccttc

SEQ ID NO: 101 - RNA, 20 nt, synthetic construct
  source 1..20: mol_type = other RNA
cctgcatctacccaccagta

SEQ ID NO: 102 - RNA, 20 nt, synthetic construct
  source 1..20: mol_type = other RNA
agctttggggaccacaattc

SEQ ID NO: 103 - RNA, 20 nt, synthetic construct
  source 1..20: mol_type = other RNA
ggccattggcaaggtaagtg

SEQ ID NO: 104 - RNA, 20 nt, synthetic construct
  source 1..20: mol_type = other RNA
aagatcaggccttctatcct

SEQ ID NO: 105 - RNA, 20 nt, synthetic construct
  source 1..20: mol_type = other RNA
cagctttggggaccacaatt

SEQ ID NO: 106 - RNA, 20 nt, synthetic construct
  source 1..20: mol_type = other RNA
cttactggtgggtagatgca

SEQ ID NO: 107 - RNA, 20 nt, synthetic construct
  source 1..20: mol_type = other RNA
gttgaatattccttactggt

SEQ ID NO: 108 - RNA, 20 nt, synthetic construct
  source 1..20: mol_type = other RNA
ctcgaaggatgttgcagccc

SEQ ID NO: 109 - RNA, 20 nt, synthetic construct
  source 1..20: mol_type = other RNA
gcgggccacaatggcagctt

SEQ ID NO: 110 - RNA, 20 nt, synthetic construct
  source 1..20: mol_type = other RNA
tctgagtcagcaccttggtc

SEQ ID NO: 111 - RNA, 20 nt, synthetic construct
  source 1..20: mol_type = other RNA
ccttggtctggccattggca

SEQ ID NO: 112 - RNA, 20 nt, synthetic construct
  source 1..20: mol_type = other RNA
cgggccacaatggcagcttt

SEQ ID NO: 113 - RNA, 20 nt, synthetic construct
  source 1..20: mol_type = other RNA
agttgaatattccttactgg

SEQ ID NO: 114 - RNA, 20 nt, synthetic construct
  source 1..20: mol_type = other RNA
cattggcaaggtaagtgtgg

SEQ ID NO: 115 - RNA, 20 nt, synthetic construct
  source 1..20: mol_type = other RNA
atgttgcagcccaggataga

SEQ ID NO: 116 - RNA, 20 nt, synthetic construct
  source 1..20: mol_type = other RNA
attggcaaggtaagtgtggc

SEQ ID NO: 117 - RNA, 20 nt, synthetic construct
  source 1..20: mol_type = other RNA
cagaagttgtcatcagtcag

SEQ ID NO: 118 - RNA, 20 nt, synthetic construct
  source 1..20: mol_type = other RNA
agaggcaatcatattatctc

SEQ ID NO: 119 - RNA, 20 nt, synthetic construct
  source 1..20: mol_type = other RNA
gaggcaatcatattatctca

SEQ ID NO: 120 - RNA, 20 nt, synthetic construct
  source 1..20: mol_type = other RNA
ccttgccaatggccagacca

SEQ ID NO: 121 - RNA, 20 nt, synthetic construct
  source 1..20: mol_type = other RNA
caacttctgagtcagcacct

SEQ ID NO: 122 - RNA, 20 nt, synthetic construct
  source 1..20: mol_type = other RNA
ggtccccaaagctgccattg

SEQ ID NO: 123 - RNA, 20 nt, synthetic construct
  source 1..20: mol_type = other RNA
cagcaccttggtctggccat

SEQ ID NO: 124 - RNA, 20 nt, synthetic construct
  source 1..20: mol_type = other RNA
attctcaaagtcagcatgga

SEQ ID NO: 125 - RNA, 20 nt, synthetic construct
  source 1..20: mol_type = other RNA
actggtgggtagatgcaggg

SEQ ID NO: 126 - RNA, 20 nt, synthetic construct
  source 1..20: mol_type = other RNA
atgtattctcaaagtcagca

SEQ ID NO: 127 - RNA, 20 nt, synthetic construct
  source 1..20: mol_type = other RNA
gggccacaatggcagctttg

SEQ ID NO: 128 - RNA, 20 nt, synthetic construct
  source 1..20: mol_type = other RNA
ttttcatctttctcattgct

SEQ ID NO: 129 - RNA, 20 nt, synthetic construct
  source 1..20: mol_type = other RNA
tagatctatgttagcgttcc

SEQ ID NO: 130 - RNA, 20 nt, synthetic construct
  source 1..20: mol_type = other RNA
catctcagagctggttcggc

SEQ ID NO: 131 - RNA, 20 nt, synthetic construct
  source 1..20: mol_type = other RNA
gctcaccttctggggtatca

SEQ ID NO: 132 - RNA, 20 nt, synthetic construct
  source 1..20: mol_type = other RNA
acgctaacatagatctagaa

SEQ ID NO: 133 - RNA, 20 nt, synthetic construct
  source 1..20: mol_type = other RNA
gctggttcggctggccatgc

SEQ ID NO: 134 - RNA, 20 nt, synthetic construct
  source 1..20: mol_type = other RNA
tagcgttcctggatccaaga

SEQ ID NO: 135 - RNA, 20 nt, synthetic construct
  source 1..20: mol_type = other RNA
ggctcaccttctggggtatc

SEQ ID NO: 136 - RNA, 20 nt, synthetic construct
  source 1..20: mol_type = other RNA
ctgatctgctgccaattacc

SEQ ID NO: 137 - RNA, 20 nt, synthetic construct
  source 1..20: mol_type = other RNA
cttgacctgcccctggtaat

SEQ ID NO: 138 - RNA, 20 nt, synthetic construct
  source 1..20: mol_type = other RNA
gatctgctgccaattaccag

SEQ ID NO: 139 - RNA, 20 nt, synthetic construct
  source 1..20: mol_type = other RNA
attaggtctggctcaccttc

SEQ ID NO: 140 - RNA, 20 nt, synthetic construct
  source 1..20: mol_type = other RNA
ttaggtctggctcaccttct

SEQ ID NO: 141 - RNA, 20 nt, synthetic construct
  source 1..20: mol_type = other RNA
caggtcaaggatgaccttct

SEQ ID NO: 142 - RNA, 20 nt, synthetic construct
  source 1..20: mol_type = other RNA
tgatctgctgccaattacca

SEQ ID NO: 143 - RNA, 20 nt, synthetic construct
  source 1..20: mol_type = other RNA
aggatgaccttcttggatcc

SEQ ID NO: 144 - RNA, 20 nt, synthetic construct
  source 1..20: mol_type = other RNA
caattaccaggggcaggtca

SEQ ID NO: 145 - RNA, 20 nt, synthetic construct
  source 1..20: mol_type = other RNA
aactcatctcagagctggtt

SEQ ID NO: 146 - RNA, 20 nt, synthetic construct
  source 1..20: mol_type = other RNA
aacatccctgataccccaga

SEQ ID NO: 147 - RNA, 20 nt, synthetic construct
  source 1..20: mol_type = other RNA
ctggtaattggcagcagatc

SEQ ID NO: 148 - RNA, 20 nt, synthetic construct
  source 1..20: mol_type = other RNA
tgctgccaattaccaggggc

SEQ ID NO: 149 - RNA, 20 nt, synthetic construct
  source 1..20: mol_type = other RNA
taggtctggctcaccttctg

SEQ ID NO: 150 - RNA, 20 nt, synthetic construct
  source 1..20: mol_type = other RNA
gaaccagctctgagatgagt

SEQ ID NO: 151 - RNA, 20 nt, synthetic construct
  source 1..20: mol_type = other RNA
aaccagctctgagatgagtt

SEQ ID NO: 152 - RNA, 20 nt, synthetic construct
  source 1..20: mol_type = other RNA
ggtcatccttgacctgcccc

SEQ ID NO: 153 - RNA, 20 nt, synthetic construct
  source 1..20: mol_type = other RNA
tacccaactcatctcagagc

SEQ ID NO: 154 - RNA, 20 nt, synthetic construct
  source 1..20: mol_type = other RNA
catagatctagaaaggaaga

SEQ ID NO: 155 - RNA, 20 nt, synthetic construct
  source 1..20: mol_type = other RNA
aagcagcagaacatccagca

SEQ ID NO: 156 - RNA, 20 nt, synthetic construct
  source 1..20: mol_type = other RNA
ctcacctcagtataagtcgg

SEQ ID NO: 157 - RNA, 20 nt, synthetic construct
  source 1..20: mol_type = other RNA
gccaccaccgacttatactg

SEQ ID NO: 158 - RNA, 20 nt, synthetic construct
  source 1..20: mol_type = other RNA
acctcagtataagtcggtgg

SEQ ID NO: 159 - RNA, 20 nt, synthetic construct
  source 1..20: mol_type = other RNA
ctctccaatcggtgatcttc

SEQ ID NO: 160 - RNA, 20 nt, synthetic construct
  source 1..20: mol_type = other RNA
cattcctgaagatcaccgat

SEQ ID NO: 161 - RNA, 20 nt, synthetic construct
  source 1..20: mol_type = other RNA
gagtggttgggctctccaat

SEQ ID NO: 162 - RNA, 20 nt, synthetic construct
  source 1..20: mol_type = other RNA
caccgacttatactgaggtg

SEQ ID NO: 163 - RNA, 20 nt, synthetic construct
  source 1..20: mol_type = other RNA
atcctcacctcagtataagt

SEQ ID NO: 164 - RNA, 20 nt, synthetic construct
  source 1..20: mol_type = other RNA
ccatccatgtcatctagcag

SEQ ID NO: 165 - RNA, 20 nt, synthetic construct
  source 1..20: mol_type = other RNA
atgacatccatatagcaggg

SEQ ID NO: 166 - RNA, 20 nt, synthetic construct
  source 1..20: mol_type = other RNA
aggaatgacatccatatagc

SEQ ID NO: 167 - RNA, 20 nt, synthetic construct
  source 1..20: mol_type = other RNA
ggaatgacatccatatagca

SEQ ID NO: 168 - RNA, 20 nt, synthetic construct
  source 1..20: mol_type = other RNA
tcaggggcatacataaagat

SEQ ID NO: 169 - RNA, 20 nt, synthetic construct
  source 1..20: mol_type = other RNA
catctagcagaggagtggtt

SEQ ID NO: 170 - RNA, 20 nt, synthetic construct
  source 1..20: mol_type = other RNA
ggtggcatgaacttgaactc

SEQ ID NO: 171 - RNA, 20 nt, synthetic construct
  source 1..20: mol_type = other RNA
tctagctcctccctgctata

SEQ ID NO: 172 - RNA, 20 nt, synthetic construct
  source 1..20: mol_type = other RNA
gtggcatgaacttgaactca

SEQ ID NO: 173 - RNA, 20 nt, synthetic construct
  source 1..20: mol_type = other RNA
cctctgctagatgacatgga

SEQ ID NO: 174 - RNA, 20 nt, synthetic construct
  source 1..20: mol_type = other RNA
cactcctctgctagatgaca

SEQ ID NO: 175 - RNA, 20 nt, synthetic construct
  source 1..20: mol_type = other RNA
tcatctagcagaggagtggt

SEQ ID NO: 176 - RNA, 20 nt, synthetic construct
  source 1..20: mol_type = other RNA
caggggcatacataaagata

SEQ ID NO: 177 - RNA, 20 nt, synthetic construct
  source 1..20: mol_type = other RNA
tggcatgaacttgaactcag

SEQ ID NO: 178 - RNA, 20 nt, synthetic construct
  source 1..20: mol_type = other RNA
catgtcatctagcagaggag

SEQ ID NO: 179 - RNA, 20 nt, synthetic construct
  source 1..20: mol_type = other RNA
aggagctagaaaagaaaata

SEQ ID NO: 180 - RNA, 20 nt, synthetic construct
  source 1..20: mol_type = other RNA
ctcactgcacattgttgttg

SEQ ID NO: 181 - skipped

SEQ ID NO: 182 - RNA, 20 nt, synthetic construct
  source 1..20: mol_type = other RNA
acattgttgttgaggatgca

SEQ ID NO: 183 - RNA, 20 nt, synthetic construct
  source 1..20: mol_type = other RNA
aacaatgtgcagtgagcatg

SEQ ID NO: 184 - RNA, 20 nt, synthetic construct
  source 1..20: mol_type = other RNA
agtctgtttctttctttagg

SEQ ID NO: 185 - RNA, 20 nt, synthetic construct
  source 1..20: mol_type = other RNA
aatagtctgtttctttcttt

SEQ ID NO: 186 - RNA, 71 nt, synthetic construct
  source 1..71: mol_type = other RNA
gtagcactaaagtgcttatagtgcaggtagtgtttagttatctactgcattatgagcacttaaagtactgc

SEQ ID NO: 187 - RNA, 70 nt, synthetic construct
  source 1..70: mol_type = other RNA
gtagcactaaagtgctaagaacatcacctttgtttagttatcggtgatgttcttagcactttagtactgc

SEQ ID NO: 188 - RNA, 70 nt, synthetic construct
  source 1..70: mol_type = other RNA
gtagcactaaagtgctatggctgagagtgatgtttagttactactctcagccatagcactttagtactgc

SEQ ID NO: 189 - RNA, 84 nt, synthetic construct
  source 1..84: mol_type = other RNA
ggccagtgttgagaggcggagacttgggcaattgctggacgctgccctgggcattgcacttgtctcggtctgacagtgccggcc

SEQ ID NO: 190 - RNA, 84 nt, synthetic construct
  source 1..84: mol_type = other RNA
ggccagtgttgtcggatcttttctccagcaattgctggacgctgccctgggaattgctggagaaaagatccgacagtgccggcc

SEQ ID NO: 191 - RNA, 82 nt, synthetic construct
  source 1..82: mol_type = other RNA
tgagctgttggattcggggccgtagcactgtctgagaggtttacatttctcacagtgaatcggtctctttttcagctgcttc

SEQ ID NO: 192 - RNA, 80 nt, synthetic construct
  source 1..80: mol_type = other RNA
tgagctgttggattggagcctattgcactgtgagaggtttacatttctcacagtgcaataggctccaattcagctgcttc

SEQ ID NO: 193 - RNA, 80 nt, synthetic construct
  source 1..80: mol_type = other RNA
tgagctgttggatggcagccttgcccactgtgagaggtttacatttctcacagtgggcaaggctgccattcagctgcttc

SEQ ID NO: 194 - RNA, 90 nt, synthetic construct
  source 1..90: mol_type = other RNA
aggcctcgctgttctctatggctttttattcctatgtgattctactgctcactcatatagggattggagccgtggcgcacggcggggaca

SEQ ID NO: 195 - RNA, 91 nt, synthetic construct
  source 1..91: mol_type = other RNA
aggcctcgctgttctcaatggcttttaagacctttggtgttctactgctcaccaccaaaggtcttaaaagccattgcgcacggcggggaca

SEQ ID NO: 196 - RNA, 90 nt, synthetic construct
  source 1..90: mol_type = other RNA
aggcctcgctgttctctatggctgagagtgactgaccattctactgctcacttggtcagtcactctcagccatagcgcacggcggggaca

SEQ ID NO: 197 - RNA, 99 nt, synthetic construct
  source 1..99: mol_type = other RNA
ccgatgtgtatcctcagctttgagaactgaattccatgggttgtgtcagtgtcagacctctgaaattcagttcttcagctgggatatctctgtcatcgt

SEQ ID NO: 198 - RNA, 98 nt, synthetic construct
  source 1..98: mol_type = other RNA
ccgatgtgtatcctcagtggacactcactttttcagagattgtgtcagtgtcagatctctgaaaaagtgagtgtccactgggatatctctgtcatcgt

SEQ ID NO: 199 - RNA, 69 nt, synthetic construct
  source 1..69: mol_type = other RNA
gggatactcaaaatgggggcgctttcctttttgtctgtactgggaagtgcttcgattttggggtgtccc

SEQ ID NO: 200 - RNA, 70 nt, synthetic construct
  source 1..70: mol_type = other RNA
gggtgaggtgatgttcttagcacttacctttttgtctgtactggtaagtgctaagaacatcacctcaccc

SEQ ID NO: 201 - RNA, 70 nt, synthetic construct
  source 1..70: mol_type = other RNA
gggatgcactgcagctgccagcactacctttttgtctgtactggtagtgctggcagctgcagtgaggccc

SEQ ID NO: 202 - RNA, 70 nt, synthetic construct
  source 1..70: mol_type = other RNA
gggatgcactctcagccatagcactacctttttgtctgtactggtagtgctatggctgagagtgcctccc

SEQ ID NO: 203 - RNA, 68 nt, synthetic construct
  source 1..68: mol_type = other RNA
gggattctcaaaagtagtaaaattctttttttgtctgtactgggaattttactacttttgagagtccc

SEQ ID NO: 204 - RNA, 23 nt, synthetic construct
  source 1..23: mol_type = other RNA
taaagtgcttatagtgcaggtag

SEQ ID NO: 205 - RNA, 23 nt, synthetic construct
  source 1..23: mol_type = other RNA
taaagtgctaagaacatcacctt

SEQ ID NO: 206 - RNA, 23 nt, synthetic construct
  source 1..23: mol_type = other RNA
taaagtgctatggctgagagtga

SEQ ID NO: 207 - RNA, 22 nt, synthetic construct
  source 1..22: mol_type = other RNA
cattgcacttgtctcggtctga

SEQ ID NO: 208 - RNA, 22 nt, synthetic construct
  source 1..22: mol_type = other RNA
aattgctggagaaaagatccga

SEQ ID NO: 209 - RNA, 21 nt, synthetic construct
  source 1..21: mol_type = other RNA
tcacagtgaatcggtctcttt

SEQ ID NO: 210 - RNA, 21 nt, synthetic construct
  source 1..21: mol_type = other RNA
tcacagtgcaataggctccaa

SEQ ID NO: 211 - RNA, 21 nt, synthetic construct
  source 1..21: mol_type = other RNA
tcacagtgggcaaggctgcca

SEQ ID NO: 212 - RNA, 23 nt, synthetic construct
  source 1..23: mol_type = other RNA
tatggctttttattcctatgtga

SEQ ID NO: 213 - RNA, 23 nt, synthetic construct
  source 1..23: mol_type = other RNA
aatggcttttaagacctttggtg

SEQ ID NO: 214 - RNA, 22 nt, synthetic construct
  source 1..22: mol_type = other RNA
tatggctgagagtgactgacca

SEQ ID NO: 215 - RNA, 22 nt, synthetic construct
  source 1..22: mol_type = other RNA
cctctgaaattcagttcttcag

SEQ ID NO: 216 - RNA, 22 nt, synthetic construct
  source 1..22: mol_type = other RNA
tctctgaaaaagtgagtgtcca

SEQ ID NO: 217 - RNA, 23 nt, synthetic construct
  source 1..23: mol_type = other RNA
gaagtgcttcgattttggggtgt

SEQ ID NO: 218 - RNA, 22 nt, synthetic construct
  source 1..22: mol_type = other RNA
aagtgctaagaacatcacctca

SEQ ID NO: 219 - RNA, 23 nt, synthetic construct
  source 1..23: mol_type = other RNA
tagtgctggcagctgcagtgagg

SEQ ID NO: 220 - RNA, 23 nt, synthetic construct
  source 1..23: mol_type = other RNA
tagtgctatggctgagagtgcct

SEQ ID NO: 221 - RNA, 22 nt, synthetic construct
  source 1..22: mol_type = other RNA
tctcaaaagtagtaaaattctt

(19) United States
(12) Patent Application Publication
(10) Pub. No.: US 2026/0014201 A1
(43) Pub. Date: Jan. 15, 2026

(54) TXNIP INHIBITION TO ENHANCE ADOPTIVE IMMUNOTHERAPY

(71) Applicant: H. Lee Moffitt Cancer Center and Research Institute, Inc., Tampa, FL (US)

(72) Inventors: Daniel Abate-Daga, Tampa, FL (US); Miguel Gomez Fontela, Thousand Oaks, CA (US)

(21) Appl. No.: 19/332,980

(22) Filed: Sep. 18, 2025

Related U.S. Application Data
(63) Continuation-in-part of application No. PCT/US2024/031279, filed on May 28, 2024.
(60) Provisional application No. 63/505,241, filed on May 31, 2023.

(51) Int. Cl. A61K 35/17; A61K 31/517; A61K 40/31; A61K 40/32; A61P 35/00; C07K 14/725; C07K 16/28; C07K 16/30; C12N 9/22; C12N 15/11; C12N 15/113; C12N 15/85

(52) U.S. Cl. CPC A61K 35/17; A61K 31/517; A61K 40/31; A61K 40/32; A61P 35/00; C07K 14/7051; C07K 16/2818; C07K 16/2827; C07K 16/3069; C12N 9/226; C12N 15/111; C12N 15/113; C12N 15/85; A61K 2239/13; A61K 2239/21; A61K 2239/22; C12N 2310/11; C12N 2310/141; C12N 2310/20

(57) ABSTRACT

Disclosed herein is a method for enhancing adoptively transferred autologous or allogeneic immune effector T-cells (including gamma delta T cells (γδ-T cells)) by targeting the thioredoxin (TRX)-interacting protein (TXNIP). In addition, disclosed herein are cDNA sequences for the co-expression of immune receptors (CARs, TCRs, etc) in combination with guide RNAs, and/or artificial or natural microRNAs, and/or shRNAs targeting TXNIP.

### CROSS-REFERENCE TO RELATED APPLICATIONS

This application is a continuation-in-part of copending International Application No. PCT/US2024/031279, filed May 28, 2024, which claims benefit of U.S. Provisional Application No. 63/505,241, filed May 31, 2023, which are hereby incorporated herein by reference in their entireties.

### STATEMENT OF GOVERNMENT INTEREST

This invention was made with Government Support under Grant No. CA241169 awarded by the National Institutes of Health. The Government has certain rights in the invention.

### SEQUENCE LISTING

This application contains a sequence listing filed in ST.26 format entitled “MOF 23MA013 CIP US Sequence Listing” created on September 16, having 195,888 bytes. The content of the sequence listing is incorporated herein in its entirety.

### BACKGROUND OF THE INVENTION

Several factors are associated with CAR T efficacy. Proliferative capacity and asymmetric division of memory and naïve like phenotypes is required to supply enough cells to eradicate tumor cells and mediate disease remission. Greater persistence results in a reservoir of tumor specific T cells to surveil for disease over time increasing durability of response. Greater metabolic fitness allows CAR T cells to perform cytolytic and secretory function in a nutrient depleted microenvironment under high oxidative stress. The cells produce ATP via oxidative metabolism to slow or prevent differentiation toward terminally differentiated or exhausted effector phenotypes. The cells also have reduced glycolytic flux. Persistent memory cells are characterized as having increased mitochondrial biomass with tubular morphology and a greater use of oxidative metabolism that relies more on the TCA cycle and ETC to produce ATP.

The percentage of central memory T cells or CCR7+CD45RO+ cells in peripheral blood after infusion also corelates with complete and durable responses. Central memory T cells are poised to proliferate and substantially contribute to peak expansion of CAR T cells after infusion and are therefore considered most desirable for therapy efficacy. Central memory T cells rely heavily on fatty acid oxidation and oxidative phosphorylation in mitochondria to synthesize ATP requiring augmented metabolic fitness. However, central memory T cells have a reduced capacity to lyse target cells relative to effector and effector memory CAR T cells. Modifying CAR T cells to increase their propensity to achieve central memory phenotypes is expected to contribute to improved therapy efficacy. However, there is a critical balance between metabolic fitness that must be targeted without sacrificing cytotoxic potential.

### SUMMARY OF THE INVENTION

Disclosed herein is a method for enhancing adoptively transferred autologous or allogeneic immune effector T-cells (including gamma delta T cells (γδ-T cells)) by targeting the thioredoxin (TRX)-interacting protein (TXNIP). In addition, disclosed herein are cDNA sequences for the co-expression of immune receptors (CARs, TCRs, etc) in combination with guide RNAs, and/or artificial or natural microRNAs, and/or shRNAs targeting TXNIP. Thioredoxins have an essential role in limiting oxidative stress, either directly or indirectly by forming a conserved reduction-oxidation (redox) signal complex with TXNIP, called redoxisome. The redoxisome constitutes a conserved regulator for redox-related signal transduction in many cell types among several species. Furthermore, TXNIP has been proposed, in a redoxisome-independent manner, as i) a master regulator for the glucose metabolism, ii) a proinflammatory element that binds to NLRP3 and triggers the inflammasome, and iii) a facilitator of protein ubiquitination through its interaction with ITCH.

Chimeric antigen receptor (CAR) T cells are engineered T cells that use a recombinant receptor for target antigen recognition and exert an antitumor response in a major histocompatibility complex (MHC)-unrestricted manner. Recent evidence suggests that the metabolic reprogramming and demands inside the tumor microenvironment (TME) negatively affects the activity of the T cells infiltrating the tumor. The improvement of the metabolic fitness of T cells has been shown to enhance their presence and antitumoral activities in the TME. TXNIP plays an important role in various types of immune cells, being required for the maintenance of the immune cell function. Due to the role of TXNIP as a master regulator of the glucose metabolism, and its increased expression in T cell subsets used for immunotherapies, we proposed new strategies to optimize CAR-T cell metabolic fitness by modulating TXNIP expression and/or activity ex vivo, during CAR-T cell manufacturing, and/or in vivo, as part of a combination treatment.

Disclosed herein are the strategies to achieve a metabolic fitness enhancement of CAR-T cells through i) genomic ablation of TXNIP expression; ii) interference RNA (RNAi) targeting the TXNIP mRNA processing; iv) inhibition of TXNIP activity and function by natural products and/or synthetic drugs; v) genomic interventions to modify regulatory elements or biologically active sites within the coding region of TXNIP, that do not result in complete ablation of its expression (for example, the use of base editors to introduce mutations).

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIGS. 1A to 1E show expression of TXNIP and its association with glycolytic rates and mitochondrial mass. FIG. 1A is a Volcano plot representing differences in phosphorylation sites between γδ and αβ activated CAR T cells. Phosphorylation of TXNIP tyrosine 349 induces TXNIP degradation. FIG. 1B shows TXNIP expression in γδ and αβ T cells, as analyzed by flow cytometry. FIG. 1C contains histograms representing the expression of GLUT-1 (left) and the mitochondrial mass (right, as measured by MitoTracker Deep Red, MTDR) of γδ and αβ T cells. FIG. 1D shows SeaHorse analysis of glycolytic activity (as extracellular acidification rate) by γδ and αβ T cells over time, after inhibition of the mitochondria (Rot/AA), and after glycolysis inhibition (2DG). FIG. 1E shows basal (left) and compensatory (right) glycolysis of resting and activated γδ and αβ CAR T cells as measured by SeaHorse analysis of proton efflux rate (statistical test: 2-way ANOVA with Tukey's multiple comparisons).

FIGS. 2A to 2D show TXNIP knock out by CRISPR-cas9. FIG. 2A is a diagram of the location of the guide RNAs (Table 1) designed to ablate TXNIP, as well as the primers used to confirm genome editing by T7 Endonuclease 1 assay (T7E1). FIG. 2B is an agarose gel of the T7E1 assay products showing mismatches around the editing site. To the right, bar graph representing the quantification of DNA cleaved by CRISPR. NTC: non targeting control guide. FIG. 2C is a Western Blot confirming a reduced expression of TXNIP in the edited cells; bar graph representing the quantification of such expression normalized to GAPDH to the right. FIG. 2D contains flow cytometry histograms of TXNIP expression in edited CAR T cells (left), mitochondrial mass (middle, measured by MitoTracker DeepRed MTDR), and GLUT-1 receptor expression (right).

FIGS. 3A to 3C show TXNIP modulation by micro RNAs. FIG. 3A is a schematic representation of a viral vector for the expression of an immune receptor (for instance, a CAR) as well as a TXNIP-targeting miRNA within a synthetic intron. Ψ represents the virus encapsidation signal. FIG. 3B, left contains flow cytometry histograms of CAR expression in αβ T cells transduced with a PSCA-specific CAR, with or without a miRNA (miR-146a or miR-128). To the right, expression of TXNIP in T cells modified with the miRNA constructs. FIG. 3C contains bar charts representing the distribution of different differentiation phenotypes in T cells expressing different CAR constructs. Representative flow cytometry dot plot with the gating strategy of untransduced T cells (UT) to the left.

FIGS. 4A to 4F show TXNIP inhibition improves metabolic traits of, and increases cytokine secretion by, T cells. FIG. 4A shows a representative example of oxygen consumption rate (OCR) of Vd2 T cells expanded in control (CTL) media or in presence of 5 uM of SRI-37330 (TXNIP inhibitor). Oli: Oligomycin, BAM15: C16H10F2N6O, Rot/AA: rotenone and antimycin A. Mean±SD. FIG. 4B shows spare respiratory capacity (SRC) of 3 independent donors. FIG. 4C shows proton efflux rate (PER) corresponding to basal glycolysis of 4 independent donors. FIG. 4D, left shows a representative example of Vd2 T cell PER; FIG. 4D, right shows compensatory glycolysis of 6 technical replicates. FIG. 4E shows IL-2 and TNFα secretion by Vd2 CAR-T cells expanded with TXNIP inhibitor versus control. Cells were co-cultured overnight with PSCA-expressing tumor cells (C42B). FIG. 4F shows TXNIP inhibition increases CAR expression. Bar chart represents the percentage of CAR+ Vd2 T cells expanded in presence or absence of SRI-37330 in the culture media, as a summary of 6 independent donors. Histograms correspond to a representative example. *p<0.05, ***p<0.001, ****p<0.0001 (t-test).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary.

It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### Thioredoxin (TRX)-Interacting Protein (TXNIP) Inhibitors

Disclosed herein are thioredoxin-interacting protein (TXNIP) inhibitors for use in the disclosed methods of enhancing adoptive immunotherapy. TXNIP is a critical regulator of oxidative stress, inflammation, and glucose metabolism, and its inhibition presents significant therapeutic potential in treating a range of diseases associated with redox imbalance and metabolic dysregulation, including diabetes mellitus, cardiovascular diseases, neurodegenerative disorders, and cancer. TXNIP functions by binding to and inhibiting thioredoxin (TRX), a key antioxidant protein, thereby promoting oxidative stress and activating downstream inflammatory pathways such as the NLRP3 inflammasome.

TXNIP inhibitors, as described herein, encompass small molecules, natural products, and nucleic acid-based therapeutics that (i) suppress TXNIP gene transcription or translation, (ii) disrupt the interaction between TXNIP and TRX, or (iii) promote proteasomal or lysosomal degradation of TXNIP protein. Examples of such inhibitors include verapamil, a calcium channel blocker shown to reduce TXNIP mRNA expression and protect pancreatic β-cells from glucotoxicity; resveratrol, a naturally occurring polyphenol that suppresses TXNIP expression and mitigates oxidative damage in neuronal and hepatic tissues; and metformin, an AMPK activator that indirectly reduces TXNIP levels and improves insulin sensitivity in metabolic disorders. Quercetin, a flavonoid compound, also downregulates TXNIP expression and has demonstrated antioxidant and anti-inflammatory effects in preclinical models. Additionally, RNA interference-based approaches, such as TXNIP-targeted siRNA and shRNA, provide direct and specific suppression of TXNIP expression and have been employed in vitro and in vivo to attenuate inflammation and cell death in models of ischemia-reperfusion injury and diabetic complications. These examples illustrate the broad applicability of TXNIP inhibition in modulating cellular redox states and inflammatory signaling. The present disclosure provides compositions and methods for inhibiting TXNIP activity or expression, alone or in combination with other therapeutic agents, to prevent or treat conditions characterized by elevated TXNIP activity or expression.

### RNAi Agents and Therapies

In some embodiments, the present invention provides a RNAi agent to TXNIP. RNAi agents to TXNIP include those compositions capable of mediating RNA interference, including, inter alia, miRNAs, shRNAs and siRNAs. In some embodiments, the RNAi agent comprises an antisense strand and a sense strand.

An embodiment of the invention provides a composition comprising an RNAi agent comprising a first (sense) or second (antisense) strand. In one embodiment, the antisense strand is about 15 to about 30 nucleotides in length, including about 19 to about 23 nucleotides in length. In one embodiment, the antisense strand has at least the length selected from about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides and 30 nucleotides.

In one embodiment, the RNAi agent comprises a modification that causes the RNAi agent to have increased stability in a biological sample or environment. In one embodiment, the RNAi agent comprises at least one sugar backbone modification (e.g., phosphorothioate linkage) or at least one 2′-modified nucleotide.

In one embodiment, the RNAi agent comprises: at least one 5′-uridine-adenine-3′ (5′-ua-3′) dinucleotide, wherein the uridine is a 2′-modified nucleotide; at least one 5′-uridine-5 guanine-3′ (5′-ug-3′) dinucleotide, wherein the 5′-uridine is a 2′-modified nucleotide; at least one 5′-cytidine-adenine-3′ (5′-ca-3′) dinucleotide, wherein the 5′-cytidine is a 2′-modified nucleotide; or at least one 5′-uridine-uridine-3′ (5′-uu-3′) dinucleotide, wherein the 5′-uridine is a 2′-modified nucleotide. These dinucleotide motifs are particularly prone to serum nuclease degradation (e.g. RNase A). Chemical modification at the 2′-position of the first pyrimidine nucleotide in the motif prevents or slows down such cleavage. This modification recipe is also known under the term ‘endo light’.

In one embodiment, the RNAi agent comprises a 2′-modification selected from the group consisting of: 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), and 2′-O—N-methylacetamido (2′-O-NMA). In one embodiment, all pyrimidines (uridine and cytidine) are 2′-O-methyl-modified nucleosides. In some embodiments, one or more nucleotides can be modified, or substituted with DNA, or a nucleotide substitute such as a peptide nucleic acid (PNA), locked nucleic acid (LNA), morpholino nucleotide, threose nucleic acid (TNA), glycol nucleic acid (GNA), arabinose nucleic acid (ANA), 2′-fluoroarabinose nucleic acid (FANA), cyclohexene nucleic acid (CeNA), anhydrohexitol nucleic acid (HNA), unlocked nucleic acid (UNA).

In one embodiment, the RNAi agent comprises at least one blunt end. In one embodiment, the RNAi agent comprises an overhang having 1 nt to 4 nt. In one embodiment, the RNAi agent comprises an overhang at the 3′-end of the antisense strand of the RNAi agent.

In one embodiment, the RNAi agent is ligated to one or more diagnostic compound, reporter group, cross-linking agent, nuclease-resistance conferring moiety, natural or unusual nucleobase, lipophilic molecule, cholesterol, lipid, lectin, steroid, uvaol, hecigenin, diosgenin, terpene, triterpene, sarsasapogenin, Friedelin, epifriedelanol-derivatized lithocholic acid, vitamin, carbohydrate, dextran, pullulan, chitin, chitosan, synthetic carbohydrate, oligo lactate 15-mer, natural polymer, low- or medium-molecular weight polymer, inulin, cyclodextrin, hyaluronic acid, protein, protein-binding agent, integrin-targeting molecule, polycationic, peptide, polyamine, peptide mimic, and/or transferrin.

RNAi agents of the present invention can be delivered or introduced (e.g., to a cell in vitro or to a subject) by any means known in the art.

“Introducing into a cell,” when referring to an iRNA, means facilitating or effecting uptake or absorption into the cell, as is understood by those skilled in the art. Absorption or uptake of an iRNA can occur through unaided diffusive or active cellular processes, or by auxiliary agents or devices. The meaning of this term is not limited to cells in vitro; an iRNA may also be “introduced into a cell,” wherein the cell is part of a living organism. In such an instance, introduction into the cell will include the delivery to the organism. For example, for in vivo delivery, iRNA can be injected into a tissue site or administered systemically. In vivo delivery can also be by a beta-glucan delivery system, such as those described in U.S. Pat. Nos. 5,032,401 and 5,607,677, and U.S. Publication No. 2005/0281781 which are hereby incorporated by reference in their entirety. In vitro introduction into a cell includes methods known in the art including, but not limited to, electroporation and lipofection. Further approaches are described below or known in the art.

Delivery of RNAi agent to tissue is a problem both because the material must reach the target organ and must also enter the cytoplasm of target cells. RNA cannot penetrate cellular membranes, so systemic delivery of naked RNAi agent is unlikely to be successful. RNA is quickly degraded by RNAse activity in serum. For these reasons, other mechanisms to deliver RNAi agent to target cells has been devised. Methods known in the art include but are not limited to: viral delivery (retrovirus, adenovirus, lentivirus, baculovirus, AAV); liposomes (Lipofectamine, cationic DOTAP, neutral DOPC) or nanoparticles (cationic polymer, PEI), bacterial delivery (tkRNAi), and also chemical modification (LNA) of siRNA to improve stability. Xia et al. 2002 Nat. Biotechnol. 20 and Devroe et al. 2002. BMC Biotechnol. 21: 15, disclose incorporation of siRNA into a viral vector. Other systems for delivery of RNAi agents are contemplated, and the RNAi agents of the present invention can be delivered by various methods yet to be found and/or approved by the FDA or other regulatory authorities.

Liposomes have been used previously for drug delivery (e.g., delivery of a chemotherapeutic). Liposomes (e.g., cationic liposomes) are described in PCT publications W002/100435A1, W003/015757A1, and WO04029213A2; U.S. Pat. Nos. 5,962,016; 5,030,453; and 6,680,068; and U.S. Patent Application 2004/0208921. A process of making liposomes is also described in W004/002453A1. Furthermore, neutral lipids have been incorporated into cationic liposomes (e.g., Farhood et al. 1995). Cationic liposomes have been used to deliver RNAi agent to various cell types (Sioud and Sorensen 2003; U.S. Patent Application 2004/0204377; Duxbury et al., 2004; Donze and Picard, 2002). Use of neutral liposomes disclosed in Miller et al. 1998, and U.S. Publ. 2003/0012812.

As used herein, the term “SNALP” refers to a stable nucleic acid-lipid particle. A SNALP represents a vesicle of lipids coating a reduced aqueous interior comprising a nucleic acid such as an iRNA or a plasmid from which an iRNA is transcribed. SNALPs are described, e.g., in U.S. Patent Application Publication Nos. 20060240093, 20070135372, and in International Application No. WO 2009082817. These applications are incorporated herein by reference in their entirety.

Chemical transfection using lipid-based, amine-based and polymer-based techniques, is disclosed in products from Ambion Inc., Austin, Tex.; and Novagen, EMD Biosciences, Inc, an Affiliate of Merck KGaA, Darmstadt, Germany); Ovcharenko D (2003) “Efficient delivery of siRNAs to human primary cells.” Ambion TechNotes 10 (5): 15-16). Additionally, Song et al. (Nat Med. published online (Fete 10, 2003) doi: 10.1038/nm828) and others [Caplen et al. 2001 Proc. Natl. Acad. Sci. (USA), 98: 9742-9747; and McCaffrey et al. Nature 414: 34-39] disclose that liver cells can be efficiently transfected by injection of the siRNA into a mammal's circulatory system.

A variety of molecules have been used for cell-specific RNAi agent delivery. For example, the nucleic acid-condensing property of protamine has been combined with specific antibodies to deliver siRNAs. Song et al. 2005 Nat Biotch. 23: 709-717. The self-assembly PEGylated polycation polyethylenimine has also been used to condense and protect siRNAs. Schiffelers et al. 2004 Nucl. Acids Res. 32: 49, 141-110.

The siRNA-containing nanoparticles were then successfully delivered to integrin overexpressing tumor neovasculature. Hu-Lieskovan et al. 2005 Cancer Res. 65: 8984-8992.

The RNAi agents of the present invention can be delivered via, for example, Lipid nanoparticles (LNP); neutral liposomes (NL); polymer nanoparticles; double-stranded RNA binding motifs (dsRBMs); or via modification of the RNAi agent (e.g., covalent attachment to the dsRNA).

Lipid nanoparticles (LNP) are self-assembling cationic lipid based systems. These can comprise, for example, a neutral lipid (the liposome base); a cationic lipid (for siRNA loading); cholesterol (for stabilizing the liposomes); and PEG-lipid (for stabilizing the formulation, charge shielding and extended circulation in the bloodstream). The cationic lipid can comprise, for example, a headgroup, a linker, a tail and a cholesterol tail. The LNP can have, for example, good tumor delivery, extended circulation in the blood, small particles (e.g., less than 100 nm), and stability in the tumor microenvironment (which has low pH and is hypoxic). Neutral liposomes (NL) are non-cationic lipid based particles. Polymer nanoparticles are self-assembling polymer-based particles. Double-stranded RNA binding motifs (dsRBMs) are self-assembling RNA binding proteins, which will need modifications.

### CRISPR

By “CRISPR” is meant a set of clustered regularly interspaced short palindromic repeats, or a system comprising such a set of repeats. By “Cas”, as used herein, is meant a CRISPR-associated protein. By “CRISPR/Cas” system is meant a system derived from CRISPR and Cas which can be used to silence or mutate the TXNIP gene.

Naturally-occurring CRISPR/Cas systems are found in approximately 40% of sequenced eubacteria genomes and 90% of sequenced archaea. Grissa et al. 2007. BMC Bioinformatics 8: 172. This system is a type of prokaryotic immune system that confers resistance to foreign genetic elements such as plasmids and phages and provides a form of acquired immunity. Barrangou et al. 2007. Science 315: 1709-1712; Marragini et al. 2008 Science 322: 1843-1845.

The CRISPR/Cas system has been modified for use in gene editing (silencing, enhancing or changing specific genes) in eukaryotes such as mice or primates. Wiedenheft et al. 2012. Nature 482: 331-8. This is accomplished by introducing into the eukaryotic cell a plasmid containing a specifically designed CRISPR and one or more appropriate Cas.

The CRISPR sequence, sometimes called a CRISPR locus, comprises alternating repeats and spacers. In a naturally-occurring CRISPR, the spacers usually comprise sequences foreign to the bacterium such as a plasmid or phage sequence; in the TXNIP CRISPR/Cas system, the spacers are derived from the TXNIP gene sequence. The repeats generally show some dyad symmetry, implying the formation of a secondary structure such as a hairpin, but they are not truly palindromic.

RNA from the CRISPR locus is constitutively expressed and processed by Cas proteins into small RNAs. These comprise a spacer flanked by a repeat sequence. The RNAs guide other Cas proteins to silence exogenous genetic elements at the RNA or DNA level. Horvath et al. 2010. Science 327: 167-170; Makarova et al. 2006 Biology Direct 1: 7. The spacers thus serve as templates for RNA molecules, analogously to siRNAs. Pennisi 2013. Science 341: 833-836.

As these naturally occur in many different types of bacteria, the exact arrangements of the CRISPR and structure, function and number of Cas genes and their product differ somewhat from species to species. Haft et al. 2005 PLoS Comput. Biol. 1: e60; Kunin et al. 2007. Genome Biol. 8: R61; Mojica et al. 2005. J. Mol. Evol. 60: 174-182; Bolotin et al. 2005. Microbiol. 151: 2551-2561; Pourcel et al. 2005. Microbiol. 151: 653-663; and Stern et al. 2010. Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. 2008. Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi 2013. Science 341: 833-836.

The CRISPR/Cas system can thus be used to edit the TXNIP gene (adding or deleting a basepair), e.g., repairing a damaged PRMT5 gene (e.g., if the damage to PRMT5 results in high post-translational modification, production, expression, level, stability or activity of TXNIP), or introducing a premature stop which thus decreases expression of an over-expressed TXNIP. The CRISPR/Cas system can alternatively be used like RNA interference, turning off the TXNIP gene in a reversible fashion. In a mammalian cell, for example, the RNA can guide the Cas protein to the TXNIP promoter, sterically blocking RNA polymerases.

Artificial CRISPR systems can be generated which inhibit TXNIP, using technology known in the art, e.g., that described in U.S. patent application Ser. No. 13/842,859 (published as US 20140068797). Such PRMT5-inhibitory CRISPR system can include a guide RNA (gRNA) comprising a PRMT5-targeting domain, i.e., a nucleotide sequence that is complementary to a TXNIP DNA strand, and a second domain that interacts with an RNA-directed nuclease, e.g., cpf1 or Cas molecule, e.g., Cas9 molecule. TABLE 1 lists exemplary sequences of a “TXNIP-targeting sequence.”

In some embodiments, the ability of an RNA-directed nuclease, e.g., cpf1 or Cas molecule, e.g., Cas9 molecule, to interact with and cleave a target nucleic acid is Protospacer Adjacent Motif (PAM) sequence dependent. A PAM sequence is a sequence in the target nucleic acid. In some embodiments, cleavage of the target nucleic acid occurs upstream from the PAM sequence. RNA-directed nuclease molecules, e.g., cpf1 or Cas molecules, e.g., Cas9 molecules, from different bacterial species can recognize different sequence motifs (e.g., PAM sequences). In addition to recognizing different PAM sequences, RNA-directed nucleases, e.g., cpf1 or Cas molecules, e.g., Cas9 molecules, from different species may be directed to different target sequences (e.g., target sequences adjacent, e.g., immediately upstream, to the PAM sequence) by gRNA molecules comprising targeting domains capable of hybridizing to said target sequences and a tracer sequence that binds to said RNA-directed nuclease, e.g., cpf1 or Cas molecule, e.g., Cas9 molecule.

In some embodiments, the CRISPR system comprises a gRNA molecule and a Cas9 molecule from S. pyogenes. A Cas9 molecule of S. pyogenes recognizes the sequence motif NGG and directs cleavage of a target nucleic acid sequence 1 to 10, e.g., 3 to 5, base pairs upstream from that sequence. A gRNA molecule useful with S. pyogenes-based CRISPR systems may include a TXNIP-targeting sequence described in TABLE 1, e.g., any of SEQ ID NOs:1-185, and a tracr sequence known to interact with S. pyogenes. See, e.g., Mali el ai, SCIENCE 2013; 339(6121): 823-826.

In some embodiments, the CRISPR system comprises a gRNA molecule and a Cas9 molecule from S. thermophilus. A Cas9 molecule of S. thermophilus recognizes a sequence motif and directs cleavage of a core target nucleic acid sequence 1 to 10, e.g., 3 to 5, base pairs upstream from these sequences. A gRNA molecule useful with S. thermophilus-based CRISPR systems may include a PRMT5-targeting sequence described in TABLE 1, e.g., any of SEQ ID NOs:1-185, and a tracr sequence known to interact with S. thermophilus. See, e.g., Horvath et al., SCIENCE 2010; 327(5962): 167-170, and Deveau et al., J BACTERIOL 2008; 190(4): 1390-1400.

In some embodiments, the CRISPR system comprises a gRNA molecule and a Cas9 molecule from S. aureus. A Cas9 molecule of S. aureus recognizes the sequence motif NNGRR (R=A or G) and directs cleavage of a target nucleic acid sequence 1 to 10, e.g., 3 to 5, base pairs upstream from that sequence. A gRNA molecule useful with S. aureus-based CRISPR systems may include a PRMT5-targeting sequence described in TABLE 1, e.g., any of SEQ ID NOs:1-185, and a tracr sequence known to interact with S. aureus. See, e.g., Ran F. et al., NATURE, vol. 520, 2015, pp. 186-191.

In some embodiments, the CRISPR system comprises a gRNA molecule and a RNA-directed nuclease, e.g., cpf1 molecule, e.g., a cpf1 molecule from L. bacterium or a cpf1 molecule from A. sp. A cpf1 molecule, e.g., a cpf1 molecule from L. bacterium or a cpf1 molecule from A. sp., recognizes a sequence motive and directs cleavage of a target nucleic acid sequence 1-25 base pairs upstream of the PAM sequence, e.g., 18-19 base pairs upstream from the PAM sequence on the same strand as the PAM and 23 base pairs upstream of the PAM sequence on the opposite strand as the PAM, creating a sticky end break. A gRNA molecule useful with cpf1-based CRISPR systems (e.g., those utilizing cpf1 molecules from L. bacterium or A. sp.) may include a PRMT5-targeting sequence described in TABLE 1, e.g., any of SEQ ID NOs:1-185, and a tracr sequence which interacts with cpf1. See, e.g., Zetsche B. et al., CELL, vol. 163:3, October 2015, 759-771.

TABLE 1
gRNA Analysis

| | | | | Specificity | Efficiency | |
|---|---|---|---|---|---|---|
| | | | | Score | Score | |
| Region | | | | (Off target | (On target | |
| targeted | Strand | Sequence (5′-3′) | PAM | score) | score) | gRNA ref# |
| Exon 1 | − | TTACTCGTGTCAAAGCCGTT | AGG | 94.4940249 | 56.69405263 | TXNIP1.1 |
| | | (SEQ ID NO: 1) | | | | |
| | + | GAAGCGTGTCTTCATAGCGC | AGG | 92.0482499 | 54.85768373 | TXNIP1.2 |
| | | (SEQ ID NO: 2) | | | | |
| | + | CTTTAGCCACTCCGCAAGCC | AGG | 87.8740432 | 48.25764733 | TXNIP1.3 |
| | | (SEQ ID NO: 3) | | | | |
| | − | CCTGAAAAGGTGTACGGCAG | TGG | 85.6652366 | 50.88626772 | TXNIP1.4 |
| | | (SEQ ID NO: 4) | | | | |
| | + | CCACTGCCGTACACCTTTTC | AGG | 84.291897 | 0.228007279 | TXNIP1.5 |
| | | (SEQ ID NO: 5) | | | | |
| | − | TGTCAAAGCCGTTAGGATCC | TGG | 84.0803684 | 45.94402333 | TXNIP1.6 |
| | | (SEQ ID NO: 6) | | | | |
| | + | tgggccgcttacCTGTTGGC | TGG | 83.9169573 | 48.14731259 | TXNIP1.7 |
| | | (SEQ ID NO: 7) | | | | |
| | + | gaattgggccgcttacCTGT | TGG | 80.3586099 | 55.71134354 | TXNIP1.8 |
| | | (SEQ ID NO: 8) | | | | |
| | + | CACTGCCGTACACCTTTTCA | GGG | 79.917685 | 37.51262168 | TXNIP1.9 |
| | | (SEQ ID NO: 9) | | | | |
| | − | TAGGATCCTGGCTTGCGGAG | TGG | 79.3329856 | 60.71168055 | TXNIP1.10 |
| | | (SEQ ID NO: 10) | | | | |
| | − | GGTGTAGGGCAGTGGCGAGA | AGG | 78.9167295 | 54.96953715 | TXNIP1.11 |
| | | (SEQ ID NO: 11) | | | | |
| | − | GCGGAGTGGCTAAAGTGCTT | TGG | 78.8123219 | 43.50432532 | TXNIP1.12 |
| | | (SEQ ID NO: 12) | | | | |
| | + | CCGAAGTCTGTTTGCACTGC | TGG | 77.4227776 | 29.40975926 | TXNIP1.13 |
| | | (SEQ ID NO: 13) | | | | |
| | − | GGCTGGCCGGGTGATAGTGG | AGG | 77.2404437 | 55.33660734 | TXNIP1.14 |
| | | (SEQ ID NO: 14) | | | | |
| | − | AACGACCCTGAAAAGGTGTA | CGG | 76.9800555 | 60.05664182 | TXNIP1.15 |
| | | (SEQ ID NO: 15) | | | | |
| | − | GGTGGCTGGCCGGGTGATAG | TGG | 76.1607752 | 42.80395119 | TXNIP1.16 |
| | | (SEQ ID NO: 16) | | | | |
| | + | TCCGCAAGCCAGGATCCTAA | CGG | 75.3249652 | 54.85440863 | TXNIP1.17 |
| | | (SEQ ID NO: 17) | | | | |
| | + | CGAAGTCTGTTTGCACTGCT | GGG | 73.3673521 | 52.78096671 | TXNIP1.18 |
| | | (SEQ ID NO: 18) | | | | |
| | + | TCACCATGATGGAACTGAGT | TGG | 72.944479 | 59.33968161 | TXNIP1.19 |
| | | (SEQ ID NO: 19) | | | | |
| | − | GCCGTTAGGATCCTGGCTTG | CGG | 71.6604792 | 63.8267222 | TXNIP1.20 |
| | | (SEQ ID NO: 20) | | | | |
| | − | GGTCTTTAACGACCCTGAAA | AGG | 70.3755299 | 44.4193638 | TXNIP1.21 |
| | | (SEQ ID NO: 21) | | | | |
| | − | CTATGAAGACACGCTTCTTC | TGG | 70.3000345 | 25.6748592 | TXNIP1.22 |
| | | (SEQ ID NO: 22) | | | | |
| | − | CTTCTGGAAGACCAGCCAAC | AGg | 68.5956574 | 54.05338892 | TXNIP1.23 |
| | | (SEQ ID NO: 23) | | | | |
| | − | GTACGGCAGTGGCGAGAAGG | TGG | 67.544025 | 60.04939651 | TXNIP1.24 |
| | | (SEQ ID NO: 24) | | | | |
| | + | CACACACCTCCACTATCACC | CGG | 67.4704527 | 64.34095612 | TXNIP1.25 |
| | | (SEQ ID NO: 25) | | | | |
| | − | GGCTAAAGTGCTTTGGATGC | AGG | 66.4970329 | 41.78955686 | TXNIP1.26 |
| | | (SEQ ID NO: 26) | | | | |
| | − | AAACCAACTCAGTTCCATCA | TGG | 63.0288329 | 61.28943665 | TXNIP1.27 |
| | | (SEQ ID NO: 27) | | | | |
| | − | GCTAAAGTGCTTTGGATGCA | GGG | 60.3930492 | 56.92685064 | TXNIP1.28 |
| | | (SEQ ID NO: 28) | | | | |
| | + | CTTCTTGAACATCACCATGA | TGG | 59.9895322 | 55.6128747 | TXNIP1.29 |
| | | (SEQ ID NO: 29) | | | | |
| | − | AAGACCAGCCAACAGgtaag | cgg | 59.35458 | 56.85594756 | TXNIP1.30 |
| | | (SEQ ID NO: 30) | | | | |
| | − | GAAGATCAAGTCTTTTGAGG | TGG | 56.9321942 | 60.28529917 | TXNIP1.31 |
| | | (SEQ ID NO: 31) | | | | |
| | − | CCAGCAGTGCAAACAGACTT | CGG | 55.7357914 | 58.79915909 | TXNIP1.32 |
| | | (SEQ ID NO: 32) | | | | |
| | − | CAAGAAGATCAAGTCTTTTG | AGG | 50.9013915 | 41.0549067 | TXNIP1.33 |
| | | (SEQ ID NO: 33) | | | | |
| | − | GGCAGTGGCGAGAAGGTGGC | TGG | 47.4607686 | 47.51799574 | TXNIP1.34 |
| | | (SEQ ID NO: 34) | | | | |
| | − | GTGGCGAGAAGGTGGCTGGC | CGG | 46.5977712 | 39.4969747 | TXNIP1.35 |
| | | (SEQ ID NO: 35) | | | | |
| | − | TGGCGAGAAGGTGGCTGGCC | GGG | 46.1419108 | 46.45213789 | TXNIP1.36 |
| | | (SEQ ID NO: 36) | | | | |
| Exon 2 | − | GTTCGGCTTTGAGCTTCCTC | AGG | 73.4068219 | 35.97738822 | TXNIP2.1 |
| | | (SEQ ID NO: 37) | | | | |
| | + | ttagctgatatttacCCCTG | AGG | 68.09397 | 62.63762427 | TXNIP2.2 |
| | | (SEQ ID NO: 38) | | | | |
| | − | GAGATGGTGATCATGAGACC | TGG | 65.2132189 | 60.43284112 | TXNIP2.3 |
| | | (SEQ ID NO: 39) | | | | |
| | + | CCATCTCATTCTCACctgaa | 999 | 63.0744639 | 60.68030698 | TXNIP2.4 |
| | | (SEQ ID NO: 40) | | | | |
| | − | TTCGGCTTTGAGCTTCCTCA | GGG | 60.0456272 | 53.8187462 | TXNIP2.5 |
| | | (SEQ ID NO: 41) | | | | |
| | − | AACAAATATGAGTACAAGTT | CGG | 58.1442294 | 43.74004357 | TXNIP2.6 |
| | | (SEQ ID NO: 42) | | | | |
| | + | ACCATCTCATTCTCACctga | agg | 57.1871816 | 57.09132106 | TXNIP2.7 |
| | | (SEQ ID NO: 43) | | | | |
| | − | TCGGCTTTGAGCTTCCTCAG | GGg | 56.3918801 | 69.85099001 | TXNIP2.8 |
| | | (SEQ ID NO: 44) | | | | |
| | − | cccttcagGTGAGAATGAGA | TGG | 51.82108 | 54.34726894 | TXNIP2.9 |
| | | (SEQ ID NO: 45) | | | | |
| | + | TTGTACTCATATTTGTTTCC | AGG | 46.3970028 | 25.7205762 | TXNIP2.10 |
| | | (SEQ ID NO: 46) | | | | |
| Exon 3 | − | AATATGGGTGTGTAGACTAC | TGG | 82.2540715 | 46.85872331 | TXNIP3. 1 |
| | | (SEQ ID NO: 47) | | | | |
| | − | ATATGGGTGTGTAGACTACT | GGG | 79.3709624 | 56.46462649 | TXNIP3.2 |
| | | (SEQ ID NO: 48) | | | | |
| | + | atggatctcacCATTAAATC | AGG | 76.4028383 | 35.68721397 | TXNIP3.3 |
| | | (SEQ ID NO: 49) | | | | |
| | + | CTTGAGTTGGCTGGCTCGGG | CGG | 73.7243492 | 53.92484139 | TXNIP3.4 |
| | | (SEQ ID NO: 50) | | | | |
| | − | AATATGGGTGTGTAGACTAC | TGG | 71 | 73 | TXNIP3.5 |
| | | (SEQ ID NO: 51) | | | | |
| | − | GTGTGTAGACTACTGGGTGA | AGG | 70.3136686 | 60.9469181 | TXNIP3.6 |
| | | (SEQ ID NO: 52) | | | | |
| | − | ATATGGGTGTGTAGACTACT | GGG | 68 | 63 | TXNIP3.7 |
| | | (SEQ ID NO: 53) | | | | |
| | − | AAACTTTGAAGTAGTGGATC | TGG | 67.9561242 | 43.94695826 | TXNIP3.8 |
| | | (SEQ ID NO: 54) | | | | |
| | + | GTCTCTTGAGTTGGCTGGCT | CGG | 67.398643 | 30.13953749 | TXNIP3.9 |
| | | (SEQ ID NO: 55) | | | | |
| | − | CCTCTGGGAACATCCTTCAA | AGG | 65.3034946 | 50.59611689 | TXNIP3.10 |
| | | (SEQ ID NO: 56) | | | | |
| | + | ggatctcacCATTAAATCAG | GGG | 64.8935522 | 61.77701537 | TXNIP3.11 |
| | | (SEQ ID NO: 57) | | | | |
| | − | CTTTGAAGTAGTGGATCTGG | TGG | 64.5369117 | 66.01861399 | TXNIP3.12 |
| | | (SEQ ID NO: 58) | | | | |
| | − | TGTCAATACCCCTGATTTAA | TGg | 64.0917808 | 27.62911984 | TXNIP3.13 |
| | | (SEQ ID NO: 59) | | | | |
| | + | TCTCTTGAGTTGGCTGGCTC | GGG | 63.3081515 | 44.27473549 | TXNIP3.14 |
| | | (SEQ ID NO: 60) | | | | |
| | + | TCTTTGTCTCTTGAGTTGGC | TGG | 61.4893171 | 39.09662466 | TXNIP3.15 |
| | | (SEQ ID NO: 61) | | | | |
| | + | tggatctcacCATTAAATCA | GGG | 61.2729436 | 51.48380112 | TXNIP3.16 |
| | | (SEQ ID NO: 62) | | | | |
| | − | attttcttgacacagGCCTC | TGG | 60.5958116 | 38.70359797 | TXNIP3.17 |
| | | (SEQ ID NO: 63) | | | | |
| | − | ttttcttgacacagGCCTCT | GGG | 60.3800128 | 51.73296425 | TXNIP3.18 |
| | | (SEQ ID NO: 64) | | | | |
| | + | CCTTTGAAGGATGTTCCCAG | AGG | 60.3561499 | 69.71132357 | TXNIP3.19 |
| | | (SEQ ID NO: 65) | | | | |
| | − | GTGTGTAGACTACTGGGTGA | AGG | 50 | 70 | TXNIP3.20 |
| | | (SEQ ID NO: 66) | | | | |
| | − | aattttcattttcttgacac | agG | 45.8432307 | 49.24914844 | TXNIP3.21 |
| | | (SEQ ID NO: 67) | | | | |
| | − | CTTTGAAGTAGTGGATCTGG | TGG | 40 | 66 | TXNIP3.22 |
| | | (SEQ ID NO: 68) | | | | |
| | − | CATCCTTCAAAGGAAAATAT | GGG | 37.2831998 | 39.93400698 | TXNIP3.23 |
| | | (SEQ ID NO: 69) | | | | |
| | − | ACATCCTTCAAAGGAAAATA | TGG | 37.2492641 | 25.77158035 | TXNIP3.24 |
| | | (SEQ ID NO: 70) | | | | |
| | + | ACACCCATATTTTCCTTTGA | AGG | 33.4995265 | 20.47997449 | TXNIP3.25 |
| | | (SEQ ID NO: 71) | | | | |
| | + | TTTTTCTTTGTCTCTTGAGT | TGG | 28.94101 | 41.47348341 | TXNIP3.26 |
| | | (SEQ ID NO: 72) | | | | |
| | − | AAAGAAAAACTTTGAAGTAG | TGG | 25.6106532 | 51.84589949 | TXNIP3.27 |
| | | (SEQ ID NO: 73) | | | | |
| Exon 4 | + | GAGACAGACACCCGCCCATC | AGG | 78.9328414 | 44.44669356 | TXNIP4.1 |
| | | (SEQ ID NO: 74) | | | | |
| | + | CACAGGTGCctatatagaag | ggg | 76.0892155 | 58.52980124 | TXNIP4.2 |
| | | (SEQ ID NO: 75) | | | | |
| | + | GACACAGGTGCctatataga | agg | 75.1727916 | 43.89578859 | TXNIP4.3 |
| | | (SEQ ID NO: 76) | | | | |
| | + | ACACAGGTGCctatatagaa | ggg | 73.6522509 | 50.52674162 | TXNIP4.4 |
| | | (SEQ ID NO: 77) | | | | |
| | + | CCCATCAGGAATGAACATGC | AGG | 73.6394431 | 57.39033916 | TXNIP4.5 |
| | | (SEQ ID NO: 78) | | | | |
| | − | TCTGCTCGAATTGACAGAAA | AGG | 63.9334174 | 47.65088497 | TXNIP4.6 |
| | | (SEQ ID NO: 79) | | | | |
| | − | AGgtaaaatcctagtgctta | tgg | 61.8828491 | 42.94925351 | TXNIP4.7 |
| | | (SEQ ID NO: 80) | | | | |
| | − | GCATGTTCATTCCTGATGGG | CGG | 61.507678 | 63.96928728 | TXNIP4.8 |
| | | (SEQ ID NO: 81) | | | | |
| | − | CATGTTCATTCCTGATGGGC | GGG | 60.9544966 | 52.49324526 | TXNIP4.9 |
| | | (SEQ ID NO: 82) | | | | |
| | − | CCTGCATGTTCATTCCTGAT | GGG | 52.077312 | 45.40025504 | TXNIP4.10 |
| | | (SEQ ID NO: 83) | | | | |
| | − | TCCTGCATGTTCATTCCTGA | TGG | 51.7850016 | 42.75580766 | TXNIP4.11 |
| | | (SEQ ID NO: 84) | | | | |
| | + | TTTTCTTTTTTAGCAGACAC | AGG | 50.2922992 | 50.15759264 | TXNIP4.12 |
| | | (SEQ ID NO: 85) | | | | |
| | − | GACAGAAAAGGATTCTGTGA | AGg | 47.9790039 | 62.5797406 | TXNIP4.13 |
| | | (SEQ ID NO: 86) | | | | |
| Exon 5 | + | GTAAGTGTGGGGGGCCACAA | TGG | 82 | 57 | TXNIP5.1 |
| | | (SEQ ID NO: 87) | | | | |
| | − | GAATACATGTTCCCGAATTG | TGG | 89.231231 | 53.96994618 | TXNIP5.2 |
| | | (SEQ ID NO: 88) | | | | |
| | + | GCCTGATCTTCTGAACCCGA | AGG | 87.3264922 | 61.96900346 | TXNIP5.3 |
| | | (SEQ ID NO: 89) | | | | |
| | + | GTAAGTGTGGGGGCCACAA | TGG | 86.8255934 | 56.60794612 | TXNIP5.4 |
| | | (SEQ ID NO: 90) | | | | |
| | − | GGGACATGCGCATCATGGCG | TGG | 85.550764 | 57.47510248 | TXNIP5.5 |
| | | (SEQ ID NO: 91) | | | | |
| | + | GTAAGGAATATTCAACTCGA | AGG | 83.0113595 | 61.5585458 | TXNIP5.6 |
| | | (SEQ ID NO: 92) | | | | |
| | − | CCTTACTGgtgggtagatgc | agg | 82.6174315 | 44.41914555 | TXNIP5.7 |
| | | (SEQ ID NO: 93) | | | | |
| | − | TCTCAGGGACATGCGCATCA | TGG | 81.5988726 | 59.55598677 | TXNIP5.8 |
| | | (SEQ ID NO: 94) | | | | |
| | − | CGCCACACTTACCTTGCCAA | TGG | 79.6053449 | 60.35193038 | TXNIP5.9 |
| | | (SEQ ID NO: 95) | | | | |
| | − | GCCTTCGGGTTCAGAAGATC | AGG | 78.8532903 | 45.38753719 | TXNIP5.10 |
| | | (SEQ ID NO: 96) | | | | |
| | − | GAAGATCAGGCCTTCTATCC | TGG | 78.1737736 | 37.33115569 | TXNIP5.11 |
| | | (SEQ ID NO: 97) | | | | |
| | − | CATGGCGTGGCAAGAGCCTT | CGG | 75.9691707 | 36.99607473 | TXNIP5.12 |
| | | (SEQ ID NO: 98) | | | | |
| | − | TCGAGTTGAATATTCCTTAC | TGg | 74.9092191 | 26.36683036 | TXNIP5.13 |
| | | (SEQ ID NO: 99) | | | | |
| | − | ATGGCGTGGCAAGAGCCTTC | GGG | 74.2746358 | 42.78418828 | TXNIP5.14 |
| | | (SEQ ID NO: 100) | | | | |
| | + | cctgcatctacccacCAGTA | AGG | 74.2062876 | 50.87381948 | TXNIP5.15 |
| | | (SEQ ID NO: 101) | | | | |
| | + | AGCTTTGGGGACCACAATTC | GGG | 72.9782639 | 38.40641296 | TXNIP5.16 |
| | | (SEQ ID NO: 102) | | | | |
| | + | GGCCATTGGGAAGGTAAGTG | TGG | 72.4571474 | 59.63784171 | TXNIP5.17 |
| | | (SEQ ID NO: 103) | | | | |
| | − | AAGATCAGGCCTTCTATCCT | GGG | 72.0637652 | 47.64928818 | TXNIP5.18 |
| | | (SEQ ID NO: 104) | | | | |
| | + | CAGCTTTGGGGACCACAATT | CGG | 71.0185455 | 31.00432354 | TXNIP5.19 |
| | | (SEQ ID NO: 105) | | | | |
| | − | CTTACTGgtgggtagatcca | ggg | 70.0785773 | 56.78689112 | TXNIP5.20 |
| | | (SEQ ID NO: 106) | | | | |
| | − | GTTGAATATTCCTTACTGgt | ggg | 69.9785855 | 58.57082449 | TXNIP5.21 |
| | | (SEQ ID NO: 107) | | | | |
| | + | CTCGAAGGATGTTGCAGCCC | AGG | 69.3614463 | 44.06083706 | TXNIP5.22 |
| | | (SEQ ID NO: 108) | | | | |
| | + | GCGGGCCACAATGGCAGCTT | TGG | 67.06773 | 22.65465099 | TXNIP5,23 |
| | | (SEQ ID NO: 109) | | | | |
| | + | TCTGAGTCAGCACCTTGGTC | TGG | 64.5558981 | 43.89269404 | TXNIP5.24 |
| | | (SEQ ID NO: 110) | | | | |
| | + | CCTTGGTCTGGCCATTGGCA | AGG | 63.9294815 | 55.22972139 | TXNIP5.25 |
| | | (SEQ ID NO: 111) | | | | |
| | + | CGGGCCACAATGGCAGCTTT | GGG | 63.4951866 | 32.71026283 | TXNIP5.26 |
| | | (SEQ ID NO: 112) | | | | |
| | − | AGTTGAATATTCCTTACTGg | tog | 63.1355283 | 55.43846439 | TXNIP5.27 |
| | | (SEQ ID NO: 113) | | | | |
| | + | CATTGGCAAGGTAAGTGTGG | CGG | 62.355666 | 58.51969516 | TXNIP5.28 |
| | | (SEQ ID NO: 114) | | | | |
| | + | ATGTTGCAGCCCAGGATAGA | AGG | 61.9577475 | 43.95236072 | TXNIP5.29 |
| | | (SEQ ID NO: 115) | | | | |
| | + | ATTGGCAAGGTAAGTGTGGC | GGG | 61.8890661 | 49.58114997 | TXNIP5.30 |
| | | (SEQ ID NO: 116) | | | | |
| | − | CAGAAGTTGTCATCAGTCAG | AGG | 61.7835829 | 66.42683684 | TXNIP5.31 |
| | | (SEQ ID NO: 117) | | | | |
| | − | AGAGGCAATCATATTATCTC | AGG | 61.6304721 | 41.59482593 | TXNIP5.32 |
| | | (SEQ ID NO: 118) | | | | |
| | − | GAGGCAATCATATTATCTCA | GGG | 61.1645331 | 63.22146212 | TXNIP5.33 |
| | | (SEQ ID NO: 119) | | | | |
| | − | CCTTGCCAATGGCGAGACCA | AGG | 60.3894439 | 65.71720102 | TXNIP5.34 |
| | | (SEQ ID NO: 120) | | | | |
| | + | CAACTTCTGAGTCAGCACCT | TGG | 60.2375831 | 71.68856685 | TXNIP5.35 |
| | | (SEQ ID NO: 121) | | | | |
| | − | GGTCCCCAAAGGTGCCATTG | TGG | 58.9010572 | 64.33382751 | TXNIP5.36 |
| | | (SEQ ID NO: 122) | | | | |
| | + | CAGCACCTTGGTCTGGCCAT | TGG | 53.8447169 | 55.60275508 | TXNIP5.37 |
| | | (SEQ ID NO: 123) | | | | |
| | + | ATTCTCAAAGTCAGCATGGA | TGG | 52.1734477 | 52.04462077 | TXNIP5.38 |
| | | (SEQ ID NO: 124) | | | | |
| | − | ACTGgtgggtagatgcaggg | tgg | 50.1749236 | 69.11669223 | TXNIP5.39 |
| | | (SEQ ID NO: 125) | | | | |
| | + | ATGTATTCTCAAAGTCAGCA | TGG | 50.0326252 | 57.61038253 | TXNIP5.40 |
| | | (SEQ ID NO: 126) | | | | |
| | + | GGGCCAGAATGGCAGCTTTG | GGG | 49.2263459 | 59.96235282 | TXNIP5.41 |
| | | (SEQ ID NO: 127) | | | | |
| | − | ttttcatctttctcattgct | agG | 43.1551019 | 51.62547801 | TXNIP5.42 |
| | | (SEQ ID NO: 128) | | | | |
| Exon 6 | − | tagATCTATGTTAGCGTTCC | TGG | 47.463094 | 22.87358137 | TXNIP6.1 |
| | | (SEQ ID NO: 129) | | | | |
| | + | CATCTCAGAGCTGGTTCGGC | TGG | 46.2648909 | 46.96957911 | TXNIP6.2 |
| | | (SEQ ID NO: 130) | | | | |
| | + | gctcacCTTCTGGGGTATCA | GGG | 43.8314024 | 44.96168112 | TXNIP6.3 |
| | | (SEQ ID NO: 131) | | | | |
| | + | ACGCTAACATAGATctagaa | agg | 43.6005967 | 49.47854358 | TXNIP6.4 |
| | | (SEQ ID NO: 132) | | | | |
| | + | GCTGGTTCGGCTGGCCATGC | TGG | 43.5313923 | 36.74281119 | TXNIP6.5 |
| | | (SEQ ID NO: 133) | | | | |
| | − | TAGCGTTCCTGGATCCAAGA | AGG | 43.1654632 | 65.28166645 | TXNIP6.6 |
| | | (SEQ ID NO: 134) | | | | |
| | + | ggctcacCTTCTGGGGTATC | AGG | 42.9979285 | 22.816041 | TXNIP6.7 |
| | | (SEQ ID NO: 135) | | | | |
| | + | CTGATCTGCTGCCAATTACC | AGG | 42.8857683 | 38.90379706 | TXNIP6.8 |
| | | (SEQ ID NO: 136) | | | | |
| | − | CTTGACCTGCCCCTGGTAAT | TGG | 42.7364136 | 31.20022167 | TXNIP6.9 |
| | | (SEQ ID NO: 137) | | | | |
| | + | GATCTGCTGCCAATTACCAG | GGG | 42.6247274 | 71.0632793 | TXNIP6.10 |
| | | (SEQ ID NO: 138) | | | | |
| | + | attaggtctggctcacCTTC | TGG | 42.5242982 | 17.16700826 | TXNIP6.11 |
| | | (SEQ ID NO: 139) | | | | |
| | + | ttaggtctggctcacCTTCT | GGG | 41.8386903 | 32.06213673 | TXNIP6.12 |
| | | (SEQ ID NO: 140) | | | | |
| | + | CAGGTCAAGGATGACCTTCT | TGG | 41.6683816 | 39.12708773 | TXNIP6.13 |
| | | (SEQ ID NO: 141) | | | | |
| | + | TGATCTGCTGCCAATTACCA | GGG | 41.5537743 | 53.83096962 | TXNIP6.14 |
| | | (SEQ ID NO: 142) | | | | |
| | + | AGGATGACCTTCTTGGATCC | AGG | 40.9475157 | 52.90152797 | TXNIP6.15 |
| | | (SEQ ID NO: 143) | | | | |
| | + | CAATTACCAGGGGCAGGTCA | AGG | 40.1805496 | 53.96122608 | TXNIP6.16 |
| | | (SEQ ID NO: 144) | | | | |
| | + | AACTCATCTCAGAGCTGGTT | CGG | 39.816091 | 44.94444134 | TXNIP6.17 |
| | | (SEQ ID NO: 145) | | | | |
| | − | AACATCCCTGATACCCCAGA | AGg | 39.6348117 | 67.79718324 | TXNIP6.18 |
| | | (SEQ ID NO: 146) | | | | |
| | − | CTGGTAATTGGCAGCAGATC | AGG | 39.3605289 | 36.55164344 | TXNIP6.19 |
| | | (SEQ ID NO: 147) | | | | |
| | + | TGCTGCCAATTACCAGGGGC | AGG | 39.1951042 | 52.28730956 | TXNIP6.20 |
| | | (SEQ ID NO: 148) | | | | |
| | + | taggtctggctcacCTTCTG | GGG | 38.2753756 | 54.7938856 | TXNIP6.21 |
| | | (SEQ ID NO: 149) | | | | |
| | − | GAACCAGCTCTGAGATGAGT | TGG | 36.4965146 | 53.10102511 | TXNIP6.22 |
| | | (SEQ ID NO: 150) | | | | |
| | − | AACCAGCTCTGAGATGAGTT | GGG | 36.3536132 | 53.12626417 | TXNIP6.23 |
| | | (SEQ ID NO: 151) | | | | |
| | − | GGTCATCCTTGACCTGCCCC | TGG | 36.1746466 | 52.87208257 | TXNIP6.24 |
| | | (SEQ ID NO: 152) | | | | |
| | + | TACCCAACTCATCTCAGAGC | TGG | 36.1191993 | 53.42812881 | TXNIP6.25 |
| | | (SEQ ID NO: 153) | | | | |
| | + | CATAGATctagaaaggaaga | tgg | 27.3683238 | 53.79368307 | TXNIP6.26 |
| | | (SEQ ID NO: 154) | | | | |
| | − | AAGCAGCAGAACATCCAGCA | TGG | 27.130646 | 65.83392365 | TXNIP6.27 |
| | | (SEQ ID NO: 155) | | | | |
| Exon 7 | + | ctcacCTCAGTATAAGTCGG | TGG | 91.5920864 | 64.00605915 | TXNIP7.1 |
| | | (SEQ ID NO: 156) | | | | |
| | − | GCCACCACCGACTTATACTG | AGg | 90.7727939 | 65.77760737 | TXNIP7.2 |
| | | (SEQ ID NO: 157) | | | | |
| | + | acCTCAGTATAAGTCGGTGG | TGG | 88.964754 | 56.63395078 | TXNIP7.3 |
| | | (SEQ ID NO: 158) | | | | |
| | + | CTCTCCAATCGGTGATCTTC | AGG | 85.3150452 | 26.85869107 | TXNIP7.4 |
| | | (SEQ ID NO: 159) | | | | |
| | − | CATTCCTGAAGATCACCGAT | TGG | 83.350611 | 58.22824111 | TXNIP7.5 |
| | | (SEQ ID NO: 160) | | | | |
| | + | GAGTGGTTGGGCTCTCGAAT | CGG | 83.1828111 | 55.80427227 | TXNIP7.6 |
| | | (SEQ ID NO: 161) | | | | |
| | − | CACCGACTTATACTGAGgtg | agg | 82.8684796 | 65.27053427 | TXNIP7.7 |
| | | (SEQ ID NO: 162) | | | | |
| | + | atcctcacCTCAGTATAAGT | CGG | 79.3040999 | 59.38149171 | TXNIP7.8 |
| | | (SEQ ID NO: 163) | | | | |
| | + | CCATCCATGTCATCTAGCAG | AGG | 73.32847 | 60.78604517 | TXNIP7.9 |
| | | (SEQ ID NO: 164) | | | | |
| | + | ATGACATCCATATAGCAGGG | AGG | 72.7891164 | 75.10961279 | TXNIP7.10 |
| | | (SEQ ID NO: 165) | | | | |
| | + | AGGAATGACATCCATATAGC | AGG | 71.9900756 | 48.99198054 | TXNIP7.11 |
| | | (SEQ ID NO: 166) | | | | |
| | + | GGAATGACATCCATATAGCA | GGG | 69.1469352 | 64.07369746 | TXNIP7.12 |
| | | (SEQ ID NO: 167) | | | | |
| | + | TCAGGGGCATACATAAAGAT | AGG | 68.7232159 | 51.04521565 | TXNIP7.13 |
| | | (SEQ ID NO: 168) | | | | |
| | + | CATCTAGCAGAGGAGTGGTT | GGG | 68.4871238 | 55.42896252 | TXNIP7.14 |
| | | (SEQ ID NO: 169) | | | | |
| | + | GGTGGCATGAACTTGAACTC | AGG | 68.2362267 | 46.16597405 | TXNIP7.15 |
| | | (SEQ ID NO: 170) | | | | |
| | − | tctagCTCCTCCCTGCTATA | TGG | 67.6384896 | 32.61468362 | TXNIP7.16 |
| | | (SEQ ID NO: 171) | | | | |
| | + | GTGGCATGAACTTGAACTCA | GGG | 64.9402343 | 58.89679767 | TXNIP7.17 |
| | | (SEQ ID NO: 172) | | | | |
| | − | CCTCTGCTAGATGACATGGA | TGG | 64.2029357 | 58.94061287 | TXNIP7.18 |
| | | (SEQ ID NO: 173) | | | | |
| | − | CACTCCTCTGCTAGATGACA | TGG | 63.9558873 | 51.33017081 | TXNIP7.19 |
| | | (SEQ ID NO: 174) | | | | |
| | + | TCATCTAGCAGAGGAGTGGT | TGG | 63.1698212 | 47.59566573 | TXNIP7.20 |
| | | (SEQ ID NO: 175) | | | | |
| | + | CAGGGGCATACATAAAGATA | GGG | 61.5821365 | 57.52166249 | TXNIP7.21 |
| | | (SEQ ID NO: 176) | | | | |
| | + | TGGCATGAACTTGAACTCAG | GGG | 58.0085016 | 73.00264907 | TXNIP7.22 |
| | | (SEQ ID NO: 177) | | | | |
| | + | CATGTCATCTAGCAGAGGAG | TGG | 53.6216532 | 63.81728343 | TXNIP7.23 |
| | | (SEQ ID NO: 178) | | | | |
| | + | AGGAGctagaaaagaaaata | tgg | 33.500257 | 39.08251746 | TXNIP7.24 |
| | | (SEQ ID NO: 179) | | | | |
| Exon 8 | + | CTCACTGGACATTGTTGTTG | AGG | 69.8526002 | 52.83431188 | TXNIP8.1 |
| | | (SEQ ID NO: 180) | | | | |
| | + | CACATTGTTGTTGAGGATGC | AGG | 62.7347244 | 44.80298414 | TXNIP8.2 |
| | | (SEQ ID NO: 181) | | | | |
| | + | ACATTGTTGTTGAGGATGCA | GGG | 56.4959168 | 64.70813949 | TXNIP8.3 |
| | | (SEQ ID NO: 182) | | | | |
| | − | AACAATGTGCAGTGAGCATG | TGG | 55.765264 | 58.31619185 | TXNIP8.4 |
| | | (SEQ ID NO: 183) | | | | |
| | − | agtctgtttctttctttagG | TGG | 41.3463109 | 50.47785767 | TXNIP8.5 |
| | | (SEQ ID NO: 184) | | | | |
| | − | aatagtctgtttcttcttt | agG | 33.9387848 | 27.91123254 | TXNIP8.6 |
| | | (SEQ ID NO: 185) | | | | |

### MicroRNA

In some embodiments, the TXNIP inhibitor is an miRNA (or miRNA mimetic), such as miR-146a, miR-128a, miR-20a, miR-25, miR-135a, or miR-373. Therefore, in some embodiments, the miRNA has the nucleic acid sequence provided in Table 2.

An miRNA mimetic includes an miRNA has the same sequence as the native or wild type miRNA, but has a modified backbone, a modified base, and/or a 5′ or 3′ end modification. In some examples an miRNA mimetic is may less susceptible to degradation or nuclease activity. An miRNA mimic is an miRNA with at least one sequence modification and having 75% or higher sequence identity to a native or wild type miRNA and that also binds to the same mRNA(s) with similar affinity as the wild type or native miRNA. The disclosed miRNAs may also be both an miRNA mimetic and an miRNA mimic, for example, an miRNA with at least one sequence modification (e.g., 75% or higher sequence identity) to a wild type miRNA, and also having a modified backbone, base, and/or end modification.

Compositions and methods for increasing stability of nucleic acid half-life and nuclease resistance are known in the art, and can include one or more modifications or substitutions to the nucleobases, sugars, or linkages of the polynucleotide. For example, the polynucleotide can be custom synthesized to contain properties that are tailored to fit a desired use. Common modifications include, but are not limited to use of locked nucleic acids, unlocked nucleic acids (UNA's), morpholinos, peptide nucleic acids (PNA), phosphorothioate linkages, phosphonoacetate, linkages, propyne analogs, 2′-O-methyl RNA, 5-Me-dC, 2′-5′ linked phosphodiester linage, Chimeric Linkages (Mixed phosphorothioate and phosphodiester linkages and modifications), conjugation with lipid and peptides, and combinations thereof.

In some embodiment, the polynucleotide includes internucleotide linkage modifications such as phosphate analogs having achiral and uncharged intersubunit linkages (e.g., Sterchak, E. P. et al., Organic Chem., 52:4202, (1987)), or uncharged morpholino-based polymers having achiral intersubunit linkages (see, e.g., U.S. Pat. No. 5,034,506). Some internucleotide linkage analogs include morpholidate, acetal, and polyamide-linked heterocycles. Locked nucleic acids (LNA) are modified RNA nucleotides (see, for example, Braasch, et al., Chem. Biol., 8(1):1-7 (2001)). Commercial nucleic acid synthesizers and standard phosphoramidite chemistry are used to make LNAs. Other backbone and linkage modifications include, but are not limited to, phosphorothioates, peptide nucleic acids, tricyclo-DNA, decoy oligonucleotide, ribozymes, spiegelmers (containing L nucleic acids, an apatamer with high binding affinity), or CpG oligomers.

Phosphorothioates (or S-oligos) are a variant of normal DNA in which one of the nonbridging oxygens is replaced by a sulfur. The sulfurization of the internucleotide bond dramatically reduces the action of endo- and exonucleases including 5′ to 3′ and 3′ to 5′ DNA POL 1 exonuclease, nucleases S1 and P1, RNases, serum nucleases and snake venom phosphodiesterase. In addition, the potential for crossing the lipid bilayer increases. Because of these important improvements, phosphorothioates have found increasing application in cell regulation. Phosphorothioates are made by two principal routes: by the action of a solution of elemental sulfur in carbon disulfide on a hydrogen phosphonate, or by the more recent method of sulfurizing phosphite triesters with either tetraethylthiuram disulfide (TETD) or 3H-1, 2-bensodithiol-3-one 1, 1-dioxide (BDTD). The latter methods avoid the problem of elemental sulfur's insolubility in most organic solvents and the toxicity of carbon disulfide. The TETD and BDTD methods also yield higher purity phosphorothioates. (See generally Uhlmann and Peymann, 1990, Chemical Reviews 90, at pages 545-561 and references cited therein, Padmapriya and Agrawal, 1993, Bioorg. & Med. Chem. Lett. 3, 761).

Peptide nucleic acids (PNA) are molecules in which the phosphate backbone of oligonucleotides is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that is similar to oligonucleotides, but are achiral and neutrally charged molecules. Peptide nucleic acids are typically comprised of peptide nucleic acid monomers. The heterocyclic bases can be any of the standard bases (uracil, thymine, cytosine, adenine and guanine) or any of the modified heterocyclic bases described below. A PNA can also have one or more peptide or amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers), and the like. Methods for the chemical assembly of PNAs are well known. See, for example, U.S. Pat. Nos. 5,539,082, 5,527,675, 5,623,049, 5,714,331, 5,736,336, 5,773,571 and 5,786,571.

In some embodiments, the polynucleotide includes one or more chemically-modified heterocyclic bases including, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-□-D-ribofuranosyl)pyridine (2-aminopyridine), and various pyrrolo- and pyrazolopyrimidine derivatives, 4-acetylcytosine, 8-hydroxy-N-6-methyladenosine, aziridinylcytosine, 5-(carboxyhydroxylmethyl) uracil, 5-bromouracil, 5-carboxymethylaminomethyl-2-thiouracil, 5-carboxymethylaminomethyluracil, dihydrouracil, N6-isopentenyladenine, 1-methyladenine, 1-methylpseudouracil, 1-methyl guanine, 1-methylinosine, 2,2-dimethylguanine, 2-methyladenine, 2-methylguanine, 3-methylcytosine , N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, 2,6-diaminopurine, and 2′-modified analogs such as, but not limited to O-methyl, amino-, and fluoro-modified analogs. Inhibitory RNAs modified with 2′-flouro (2′-F) pyrimidines appear to have favorable properties in vitro (Chiu and Rana 2003; Harborth et al. 2003). Moreover, one report recently suggested 2′-F modified siRNAs have enhanced activity in cell culture as compared to 2′-OH containing siRNAs (Chiu and Rana 2003). 2′-F modified siRNAs are functional in mice but that they do not necessarily have enhanced intracellular activity over 2′-OH siRNAs.

In some embodiments the polynucleotide include one or more sugar moiety modifications, including, but are not limited to, 2′-O-aminoethoxy, 2′-O-amonioethyl (2′-OAE), 2′-O-methoxy, 2′-O-methyl, 2-guanidoethyl (2′-OGE), 2′-O,4′-C-methylene (LNA), 2′-O-(methoxyethyl) (2′-OME) and 2′-O—(N-(methyl)acetamido) (2′-OMA).

TABLE 2
Micro RNA sequences designed to modulate TXNIP expression.

| miRNA | | | | TXNIP |
|---|---|---|---|---|
| name | Origin | DNA Sequence (5′-3′) | Mature miRNA | target |
| miR-20a | Natural | GTAGCACTAAAGTGCTTATAGTGCAGGTAGTGT | TAAAGTGCTTATAG | 3′UTR |
| | | TTAGTTATCTACTGCATTATGAGCACTTAAAGTA | TGCAGGTAG (SEQ | |
| | | CTGC (SEQ ID NO: 186) | ID NO: 204) | |
| miR-20a | Synthetic | GTAGCACTAAAGTGCTAAGAACATCACCTTTGT | TAAAGTGCTAAGAA | 3′UTR |
| TargetA | | TTAGTTATCGGTGATGTTCTTAGCACTTTAGTAC | CATCACCTT (SEQ | |
| | | TGC (SEQ ID NO: 187) | ID NO: 205) | |
| miR-20a | Synthetic | GTAGCACTAAAGTGCTATGGCTGAGAGTGATG | TAAAGTGCTATGG | 3′UTR |
| TargetB | | TTTAGTTACTACTCTCAGCCATAGCACTTTAGTA | CTGAGAGTGA | |
| | | CTGC (SEQ ID NO: 188) | (SEQ ID NO: 206) | |
| miR-25 | Natural | GGCCAGTGTTGAGAGGCGGAGACTTGGGCAAT | CATTGCACTTGTCT | UTR |
| | | TGCTGGACGCTGCCCTGGGCATTGCACTTGTCT | CGGTCTGA (SEQ | |
| | | CGGTCTGACAGTGCCGGCC (SEQ ID NO: 189) | ID NO: 207) | |
| miR-25 | Synthetic | GGCCAGTGTTGTCGGATCTTTTCTCCAGCAATT | AATTGCTGGAGAA | UTR |
| TargetA | | GCTGGACGCTGCCCTGGGAATTGCTGGAGAAA | AAGATCCGA (SEQ | |
| | | AGATCCGACAGTGCCGGCC (SEQ ID NO: 190) | ID NO: 208) | |
| miR-128a | Natural | TGAGCTGTTGGATTCGGGGCCGTAGCACTGTCT | TCACAGTGAATCG | 3′UTR |
| | | GAGAGGTTTACATTTCTCACAGTGAATCGGTCT | GTCTCTTT (SEQ ID | |
| | | CTTTTTCAGCTGCTTC (SEQ ID NO: 191) | NO: 209) | |
| miR-128a | Synthetic | TGAGCTGTTGGATTGGAGCCTATTGCACTGTGA | TCACAGTGCAATA | 3′UTR |
| TargetA | | GAGGTTTACATTTCTCACAGTGCAATAGGCTCC | GGCTCCAA (SEQ | |
| | | AATTCAGCTGCTTC (SEQ ID NO: 192) | ID NO: 210) | |
| miR-128a | Synthetic | TGAGCTGTTGGATGGCAGCCTTGCCCACTGTG | TCACAGTGGGCAA | 3′UTR |
| TargetB | | AGAGGTTTACATTTCTCACAGTGGGCAAGGCTG | GGCTGCCA (SEQ | |
| | | CCATTCAGCTGCTTC (SEQ ID NO: 193) | ID NO: 211) | |
| miR-135a | Natural | AGGCCTCGCTGTTCTCTATGGCTTTTTATTCCTA | TATGGCTTTTTATT | 3′UTR |
| | | TGTGATTCTACTGCTCACTCATATAGGGATTGG | CCTATGTGA (SEQ | |
| | | AGCCGTGGCGCACGGCGGGGACA (SEQ ID | ID NO: 212) | |
| | | NO: 194) | | |
| miR-135a | Synthetic | AGGCCTCGCTGTTCTCAATGGCTTTTAAGACCT | AATGGCTTTTAAGA | 3′UTR |
| TargetA | | TTGGTGTTCTACTGCTCACCACCAAAGGTCTTA | CCTTTGGTG (SEQ | |
| | | AAAGCCATTGCGCACGGGGGGACA (SEQ ID | ID NO: 213) | |
| | | NO: 195) | | |
| miR-135a | Synthetic | AGGCCTCGCTGTTCTCTATGGCTGAGAGTGACT | TATGGCTGAGAGT | 3′UTR |
| TargetB | | GACCATTCTACTGCTCACTTGGTCAGTCACTCT | GACTGACCA (SEQ | |
| | | CAGCCATAGCGCACGGCGGGGACA (SEQ ID | ID NO: 214) | |
| | | NO: 196) | | |
| miR-146a | Natural | CCGATGTGTATCCTCAGCTTTGAGAACTGAATTC | CCTCTGAAATTCAG | 3′UTR |
| | | CATGGGTTGTGTCAGTGTCAGACCTCTGAAATT | TTCTTCAG (SEQ ID | |
| | | CAGTTCTTCAGCTGGGATATCTCTGTCATCGT | NO: 215) | |
| | | (SEQ ID NO: 197) | | |
| miR-146a | Synthetic | CCGATGTGTATCCTCAGTGGACACTCACTTTTT | TCTCTGAAAAAGTG | 3′UTR |
| TargetA | | CAGAGATTGTGTCAGTGTCAGATCTCTGAAAAA | AGTGTCCA (SEQ ID | |
| | | GTGAGTGTCCACTGGGATATCTCTGTCATCGT | NO: 216) | |
| | | (SEQ ID NO: 198) | | |
| miR-373 | Natural | GGGATACTCAAAATGGGGGCGCTTTCCTTTTTG | GAAGTGCTTCGATT | 3′UTR |
| | | TCTGTACTGGGAAGTGCTTCGATTTTGGGGTGT | TTGGGGTGT (SEQ | |
| | | CCC (SEQ ID NO: 199) | ID NO: 217) | |
| miR-373 | Synthetic | GGGTGAGGTGATGTTCTTAGCACTTACCTTTTT | AAGTGCTAAGAAC | 3′UTR |
| TargetA | | GTCTGTACTGGTAAGTGCTAAGAACATCACCTC | ATCACCTCA (SEQ | |
| | | ACCC (SEQ ID NO: 200) | ID NO: 218) | |
| miR-373 | Synthetic | GGGATGCACTGCAGCTGCCAGCACTACCTTTTT | TAGTGCTGGCAGC | 3′UTR |
| TargetB | | GTCTGTACTGGTAGTGCTGGCAGCTGCAGTGA | TGCAGTGAGG | |
| | | GGCCC (SEQ ID NO: 201) | (SEQ ID NO: 219) | |
| miR-373 | Synthetic | GGGATGCACTCTCAGCCATAGCACTACCTTTTT | TAGTGCTATGGCT | 3′UTR |
| TargetC | | GTCTGTACTGGTAGTGCTATGGCTGAGAGTGC | GAGAGTGCCT | |
| | | CTCCC (SEQ ID NO: 202) | (SEQ ID NO: 220) | |
| miR-373 | Synthetic | GGGATTCTCAAAAGTAGTAAAATTCTTTTTTTGT | TCTCAAAAGTAGTA | 3′UTR |
| TargetD | | CTGTACTGGGAATTTTACTACTTTTGAGAGTCC | AAATTCTT (SEQ ID | |
| | | C (SEQ ID NO: 203) | NO: 221) | |

### Small Molecules

In some embodiments, the TXNIP inhibitor is a small molecule. For example, TXNIP specific inhibitors are described in CN120483959A for use in treating osteoporosis, which is incorporated by reference in its entirety for the teaching of these compounds.

Therefore, the TXNIP inhibitor can be compound represented by the formula (I-1) or the formula (I-2) or an isotopic form, a stereoisomer, a tautomer, a cis-trans isomer, a pharmaceutically acceptable salt, a pharmaceutically acceptable solvate, a hydrate, a prodrug and a polymorph thereof,

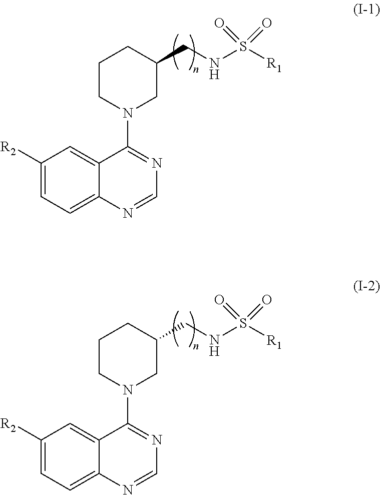

wherein, the
N is selected from any integer from 0 to 2;
R₁ is selected from one of deuterium substituted or unsubstituted C₁-C₄ alkyl, deuterium substituted or unsubstituted C₃-C₆ cycloalkyl; and
R₂ is selected from one of hydrogen, halogen, cyano, —SF₅, fluoro-substituted or unsubstituted C₁-C₄ alkyl, C₁-C₄ alkoxy, and C₃-C₆ cycloalkyl.

For example, R₁ can be —CH₃ or -CD₃, and R₂ can be trifluoromethyl.

Further, the compound is a compound represented by the following structural formulas 1 to 4: For example, the TXNIP inhibitor can be a compound represented by the following structural formulas 1 to 4:

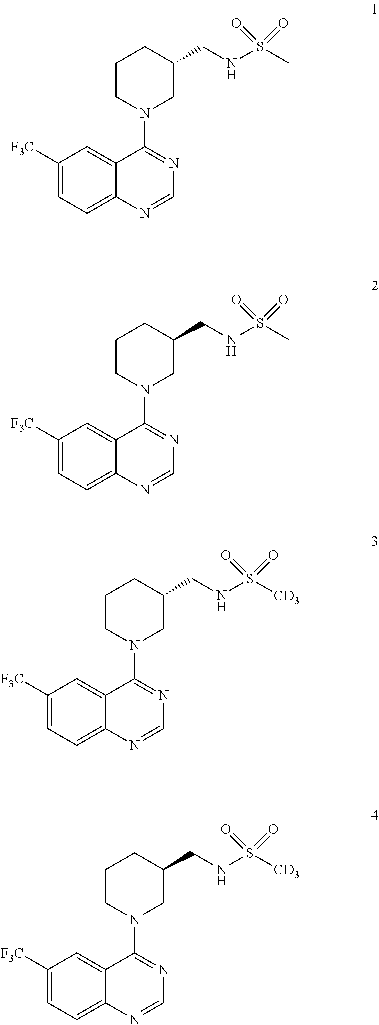 [SMILES: CS(=O)(=O)NC[C@H]1CCCN(c2ncnc3ccc(C(F)(F)F)cc23)C1 ; CS(=O)(=O)NC[C@@H]1CCCN(c2ncnc3ccc(C(F)(F)F)cc23)C1 ; [2H]C([2H])([2H])S(=O)(=O)NC[C@H]1CCCN(c2ncnc3ccc(C(F)(F)F)cc23)C1 ; [2H]C([2H])([2H])S(=O)(=O)NC[C@@H]1CCCN(c2ncnc3ccc(C(F)(F)F)cc23)C1]

As one example, SRI-37330 hydrochloride (CAS No. 2322245-49-6) is an orally bioavailable thioredoxin-interacting protein (TXNIP) inhibitor. SRI-37330 hydrochloride inhibits glucagon secretion and function, reduces hepatic glucose production and reverses hepatic steatosis. The structural formula is shown as the formula:

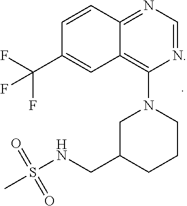 [SMILES: CS(=O)(=O)NCC1CCCN(c2ncnc3ccc(C(F)(F)F)cc23)C1]

As used herein, the term “effective amount” or “effective dose” refers to an amount that is functionally or actively (i.e., to prevent and treat diabetic osteoporosis, bone fractures, and postmenopausal osteoporosis) in a human and/or animal and that is acceptable to a human and/or animal.

As used herein, the term “pharmaceutically acceptable” ingredients are substances that are suitable for use in humans and/or mammals without undue adverse side effects (such as toxicity, irritation, and allergic response), i.e., commensurate with a reasonable benefit/risk ratio. The term “pharmaceutically acceptable carrier” refers to a carrier for administration of a therapeutic agent, including various excipients and diluents.

The pharmaceutical compositions of the present invention contain a safe and effective amount of the active ingredients of the present invention and a pharmaceutically acceptable carrier. Such carriers include, but are not limited to, saline, buffers, dextrose, water, glycerol, ethanol, gels, and combinations thereof. Generally, the pharmaceutical preparation is matched with the administration mode, and the dosage forms of the pharmaceutical composition are injection, oral preparation (tablet, capsule and oral liquid), transdermal agent and sustained release agent. For example, by using physiological saline or an aqueous solution containing glucose and other auxiliary agents by conventional methods. The pharmaceutical compositions are preferably manufactured under sterile conditions.

The effective amount of the active ingredient described herein may vary depending upon the mode of administration, the severity of the condition being treated, and the like. The selection of the preferred effective amount can be determined by one of ordinary skill in the art based on a variety of factors (e.g., by clinical trials). Such factors include, but are not limited to, the pharmacokinetic parameters of the active ingredient such as bioavailability, metabolism, half-life, etc., the severity of the disease to be treated by the patient, the weight of the patient, the immune status of the patient, the route of administration, etc. Generally, satisfactory results are obtained when the active ingredient of the present invention is administered at a dose of about 0.01 mg to 50 mg per kg of animal body weight (preferably 0.1 mg to 10 mg per kg of animal body weight) per day. For example, separate doses may be administered several times per day, or the dose may be proportionally reduced, as dictated by the urgent need for the treatment of the condition.

In some embodiments, dosage forms of TXNIP inhibitors include, but are not limited to, injection solutions, freeze-dried powder for injection, suspensions, implant I agents, embolization agents, capsules, tablets, pills, and oral liquids.

In some embodiments, the TXNIP inhibitor also comprises one or more of pharmaceutically acceptable carriers, excipients and diluents, preferably, pharmaceutically acceptable carriers include microspheres, nanoparticles and liposomes.

In some embodiments, dosage forms include, but are not limited to, injection, freeze-dried powder for injection, suspension, implant I, suppository, capsule, tablet, pill and oral liquid.

Further, the solid dosage forms may be coated or microencapsulated with enteric coating materials and/or other materials known in the art. The release of the active ingredient in such solid dosage forms may be released in a delayed manner in a certain part of the digestive tract.

### Chimeric Antigen Receptors (CAR)

In some cases, the lymphocytes also expresses a chimeric receptor. In some embodiments, the chimeric receptor comprises a chimeric antigen receptor (CAR) polypeptide.

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). The disclosed CAR is generally made up of three domains: an ectodomain, a transmembrane domain, and an endodomain. The ectodomain comprises the recognition domain. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41 BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

Additional CAR constructs are described, for example, in Fresnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41 BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., anti-CD123 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), SLAMF6 (NTB-A, Ly108), SLAM (SLAMFI, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the recognition domain is a single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target two different antigens. Also disclosed are CARs designed to work only in conjunction with another CAR that binds a different antigen, such as a tumor antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, TIM3, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCRi, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### Nucleic Acids and Vectors

Also disclosed are polynucleotides and polynucleotide vectors encoding the disclosed chimeric receptors. Also disclosed are oligonucleotides for use in inserting the chimeric receptors into the genome of a T cell at a site that will disrupt Sirt2 expression or activity.

Nucleic acid sequences encoding the disclosed chimeric receptors, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

### Immune Effector Cells

Also disclosed are immune effector cells that are engineered to express the disclosed chimeric receptors. These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T-cells are divided into two groups based on their T-Cell Receptor (TCR) components. The TCR heterodimer consists of an α and β chain in 95% of T cells. These recognize foreign antigens via peptides presented by MHC molecules on antigen presenting cells and are essential for adaptive immunity. 5% of T cells have TCRs consisting of γ and δ chains. γδ TCRs are MHC independent and detect markers of cellular stress expressed by tumors.

γδ T cells recognize pathogens and transformed cells in an HLA-unrestricted manner. They respond to markers of cellular stress (e.g. phosphoantigens released by transformed cells as by-products of the mevalonate biosynthetic pathway). γδ T cells display both innate cytotoxic functions and antigen-presenting capability, particularly in the presence of antibody-opsonized target cells.

γδ T-cells are responsible for “lymphoid stress surveillance,” i.e., sensing and responding immediately to infections or non-microbial stress without the need of clonal expansion or de novo differentiation.

The activation of γδ T cells is regulated by a balance between stimulatory and inhibitory signals. They are activated by γδ TCR ligands (e.g. phosphoantigens) in combination with MHC-associated ligands of the activatory receptor killer cell lectin-like receptor subfamily K, member 1 (KLRK1), also known as NKG2D, such as MHC class I polypeptide-related sequence A (MICA), MICB, and various members of the UL16-binding protein (ULBP) family.

In some embodiments, a γδ CAR T cell will only be fully activated and capable of killing a target cell which expresses a first antigen which is capable of binding to the γδ TCR and a second antigen which is capable of binding to the CAR. By way of example, a γδ TCR may recognize phosphoantigens (e.g. Isopentenyl pyrophosphate (IPP), Bromohydrin Pyrophosphate (BrHPP) and (E)-4-Hydroxy-3-methyl-but-2-enyl pyrophosphate (HMBPP)); major histocompatibility complex class I chain-related A (MICA); major histocompatibility complex class I chain-related B (MICB); NKG2D ligand 1-6 (ULBP 1-6); CD1c; CD1d; endothelial protein C receptor (EPCR); lipohexapeptides; phycoreythrin or histidyl-tRNA-synthase.

The disclosed system involves the use of γδ T cells that are engineered to express the disclosed CARs (also referred to herein as “γδ CAR T cells”). These cells are preferably obtained from the subject to be treated (i.e. are autologous). However, in some embodiments, cell lines or donor effector cells (allogeneic) are used.

Methods for obtaining and enriching γδ T cells are known in the art and can be used in the present methods. For example, the method can involve stimulating a mixture of T cells with a γδ T cell stimulating agent. As used herein, a “γδ T cell stimulating agent” refers to any agent which selectively stimulates the proliferation and/or survival of γδ T cells from a mixed starting population of cells. The resulting cell population is enriched with an increased number of γδ T cells. This can be achieved by stimulating fresh peripheral blood mononuclear cells (PBMC) with culture medium supplemented with zoledronate (or other amino-bisphosphonates). Retroviral transduction can be performed between days 5-7 post-stimulation, following standard protocols.

Therefore, in some embodiments, the T cells comprise γδ T cells, which possess a distinct T-cell receptor (TCR) having one γ chain and one δ chain instead of α and β chains. In some embodiments, the immune effector cells are derived from stem cells, such as induced pluripotent stem cells (IPSCs). For example, in some embodiments, the immune effector cells are γδ T cells or NK cells derived from IPSCs.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (Tc cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-1-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

### Therapeutic Methods

Immune effector cells expressing the disclosed chimeric receptors can elicit an anti-tumor immune response against cancer cells. The anti-tumor immune response elicited by the disclosed chimeric cells may be an active or a passive immune response. In addition, the immune response may be part of an adoptive immunotherapy approach in which chimeric cells induce an immune response specific to the target antigen.

Adoptive transfer of immune effector cells expressing chimeric receptors is a promising anti-cancer therapeutic. Following the collection of a patient's immune effector cells, the cells may be genetically engineered to express the disclosed chimeric receptors while ablating Sirt2 according to the disclosed methods, then infused back into the patient.

The disclosed chimeric effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat tumors. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed chimeric cells are administered to a patient in conjunction with (e.g., before, simultaneously or following) any number of relevant treatment modalities, including but not limited to thalidomide, dexamethasone, bortezomib, and lenalidomide. In further embodiments, the chimeric cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed chimeric cells can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed chimeric cells can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHlgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed chimeric cells can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1 BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent is a targeted agent, such as ibrutinib or idelalisib.

In some embodiments, such an additional therapeutic agent is an epigenetic modifier such as azacitdine or vidaza.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with chimeric cells for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C—C chemokine families. Suitable cytokines include cytokine derivatives, cytokine variants, cytokine fragments, and cytokine fusion proteins.

In some embodiments, a therapeutic agent for use in combination with chimeric cells for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with chimeric cells for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with chimeric cells for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed chimeric cells are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed chimeric cells are administered in combination with surgery.

### EMBODIMENTS

Embodiment 1. A recombinant polynucleotide comprising a
(a) a first nucleic acid sequence encoding a chimeric antigen receptor (CAR) polypeptide and/or T cell receptor; and
(b) a second nucleic acid comprising an miRNA that inhibits thioredoxin (TRX)-interacting protein (TXNIP).

Embodiment 2. The recombinant polynucleotide of embodiment 1, wherein the TXNIP inhibitor comprises a miRNA polynucleotide comprising a nucleic acid sequence encoding selected from the group consisting of SEQ ID NO:204-221.

Embodiment 3. The recombinant polynucleotide of embodiment 1 or 2, wherein the first and second nucleic acid sequences are operably linked to a single expression control sequence.

Embodiment 4. The recombinant polynucleotide of any one of embodiments 1 to 3, wherein the T cell receptor is a γδ receptor or αβ receptor.

Embodiment 5. The recombinant polynucleotide of any one of embodiments 1 to 4, wherein the first and second nucleic acid are in a viral vector.

Embodiment 6. A system comprising a
(a) a first nucleic acid sequence encoding a chimeric antigen receptor (CAR) polypeptide and/or T cell receptor; and
(b) a second nucleic acid sequence encoding a gRNA that targets a thioredoxin (TRX)-interacting protein (TXNIP); and
(c) a third nucleic acid sequence encoding a CRISPR-associated (Cas) endonuclease.

Embodiment 7. The system of embodiment 6, wherein the first, second, and third nucleic acids are in the same vector.

Embodiment 8. The system of embodiment 6 or 7, wherein the gRNA comprises a nucleic acid sequence selected from the group consisting of SEQ ID NO:1-185 and a CRISPR-associated (Cas) endonuclease.

Embodiment 9. The system of claim any one of embodiments 6 to 8, wherein the first, second, and third nucleic acid sequences are operably linked to a single expression control sequence.

Embodiment 10. The system of any one of embodiments 6 to 9, wherein the T cell receptor is a γδ receptor or αβ receptor.

Embodiment 11. The system of any one of embodiments 6 to 10, further comprising an immune effector cell expressing the first, second, and third nucleic acid sequences.

Embodiment 12. A method for enhancing adoptively transferred immune effector T-cells in a subject, comprising co-administering to the subject a thioredoxin (TRX)-interacting protein (TXNIP) inhibitor.

Embodiment 13. The method of embodiment 12, wherein the immune effector T-cells are γδ-T cells or T cells engineered to express a γδ-T cell receptor (TCR).

Embodiment 14. The method of embodiment 13, wherein the immune effector T-cells are tumor infiltrating lymphocytes (TILs).

Embodiment 15. The method of embodiment 12, wherein the immune effector T-cells are αβ-T cells or T cells engineered to express a αβ-T cell receptor (TCR).

Embodiment 16. The method of any one of embodiments 12 to 15, wherein the immune effector T-cells are autologous or allogeneic.

Embodiment 17. The method cell of any one of embodiments 12 to 16, wherein the immune effector T-cells express a chimeric antigen receptor (CAR) polypeptide.

Embodiment 18. The method cell of any one of embodiments 12 to 17, wherein the TXNIP inhibitor comprises an siRNA, antisense, or gRNA oligonucleotide.

Embodiment 19. The method of embodiment 18, wherein the TXNIP inhibitor comprises a polynucleotide comprising a nucleic acid sequence encoding a gRNA selected from the group consisting of SEQ ID NO:1-185 and a nucleic acid sequence encoding a CRISPR-associated (Cas) endonuclease.

Embodiment 20. The method of embodiment 18, wherein the TXNIP inhibitor comprises a miRNA polynucleotide comprising a nucleic acid sequence encoding selected from the group consisting of SEQ ID NO:204-221.

Embodiment 21. The method cell of any one of embodiments 12 to 20, further comprising administering to the subject a checkpoint inhibitor.

Embodiment 22. The method of embodiment 21, wherein the checkpoint inhibitor comprises an anti-PD-1 antibody, anti-PD-L1 antibody, anti-CTLA-4 antibody, or a combination thereof.

Embodiment 23. A method of providing an anti-cancer immunity in a subject, comprising
(a) administering to the subject an effective amount of a T cell expressing a gamma-delta T cell receptor (TCR) and chimeric antigen receptor (CAR) polypeptide, wherein the CAR comprises a tumor antigen binding domain, a transmembrane domain, an intracellular signaling domain, and a co-stimulatory signaling region, and
(b) administering to the subject a thioredoxin (TRX)-interacting protein (TXNIP) inhibitor.

Embodiment 24. The method of embodiment 23, wherein the TXNIP inhibitor comprises an siRNA, antisense, or gRNA oligonucleotide.

Embodiment 25. The method of embodiment 24, wherein the TXNIP inhibitor comprises a polynucleotide comprising a nucleic acid sequence encoding a gRNA selected from the group consisting of SEQ ID NO:1-185 and a nucleic acid sequence encoding a CRISPR-associated (Cas) endonuclease.

Embodiment 26. The method of embodiment 24, wherein the TXNIP inhibitor comprises a miRNA polynucleotide comprising a nucleic acid sequence encoding selected from the group consisting of SEQ ID NO:204-221.

Embodiment 27. The method of any one of embodiments 23 to 26, further comprising administering to the subject a checkpoint inhibitor.

Embodiment 28. The method of embodiment 27, wherein the checkpoint inhibitor comprises an anti-PD-1 antibody, anti-PD-L1 antibody, anti-CTLA-4 antibody, or a combination thereof.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

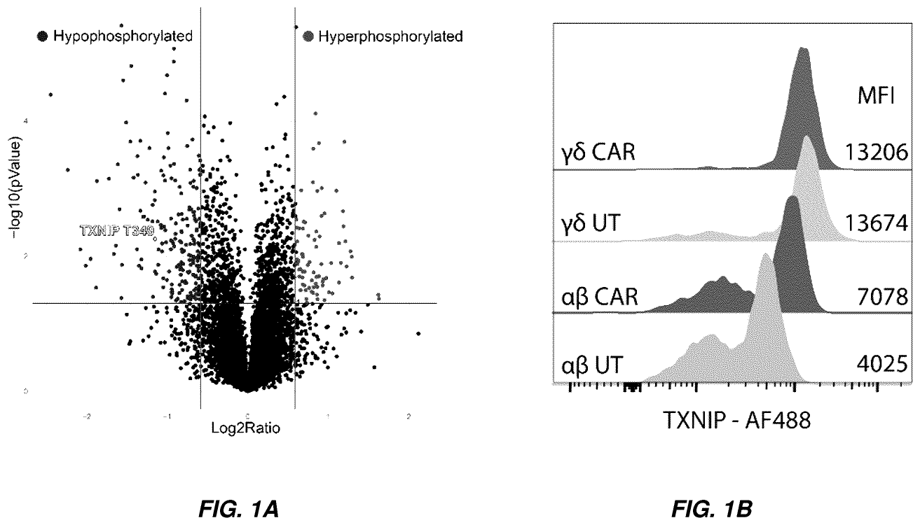

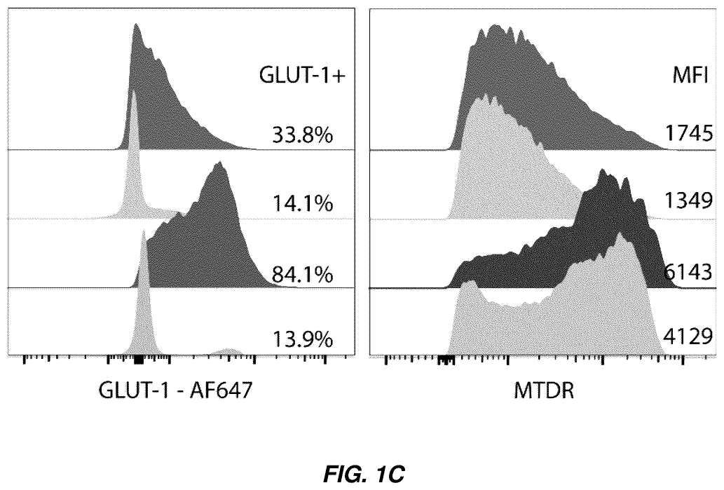

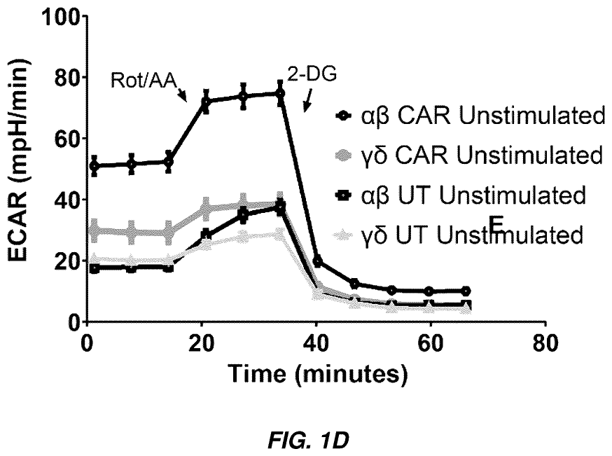

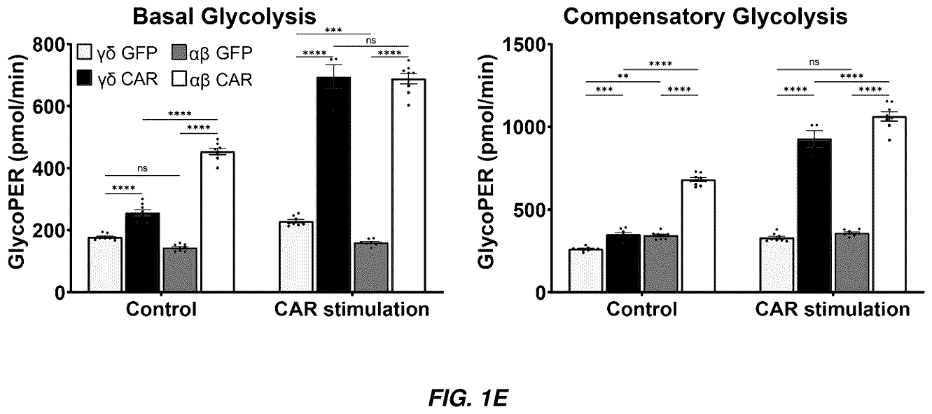

Thioredoxin-interacting protein (TXNIP) is a pleiotropic molecule that regulates metabolism and the redox balance of the cell. Among its multiple functions, TXNIP is known to inhibit the expression of glucose transporters (GLUT1), to regulate glycolysis and oxidative phosphorylation, to activate stress responses, and to trigger pyroptosis in an NLRP3 inflammasome-dependent fashion. As part of a systematic analysis of CAR signaling, where we compared the phosphorylation events triggered by a second-generation CAR (PSCA-8T28Z) in Vd2-enriched T cells versus alpha/beta T cells, we identified TXNIP as differentially phosphorylated among subsets, in response to CAR activation. This differential phosphorylation was associated with increased total expression of the TXNIP protein in Vd2 T cells and, coincidentally, with lower expression of GLUT1, lower mitochondrial content, and lower glycolysis (FIG. 1).

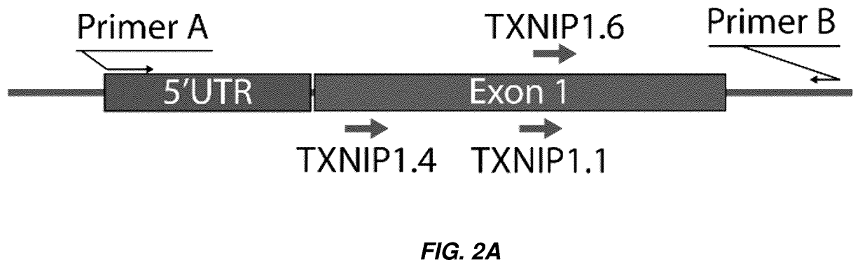

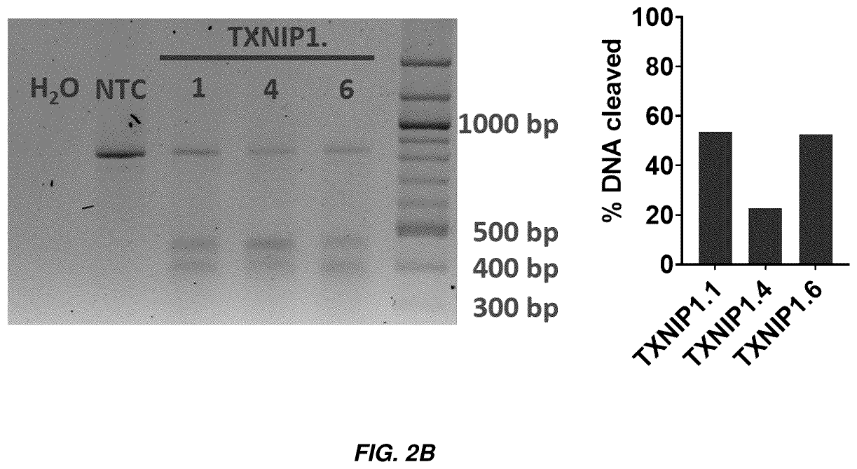

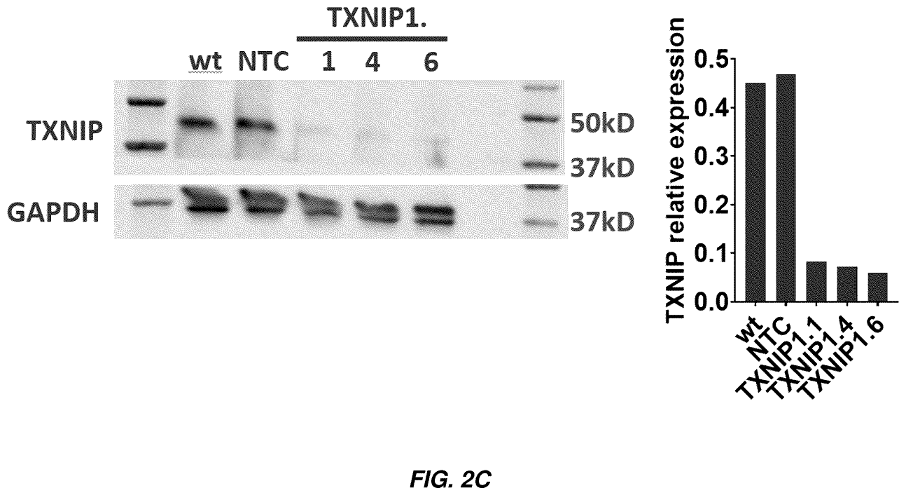

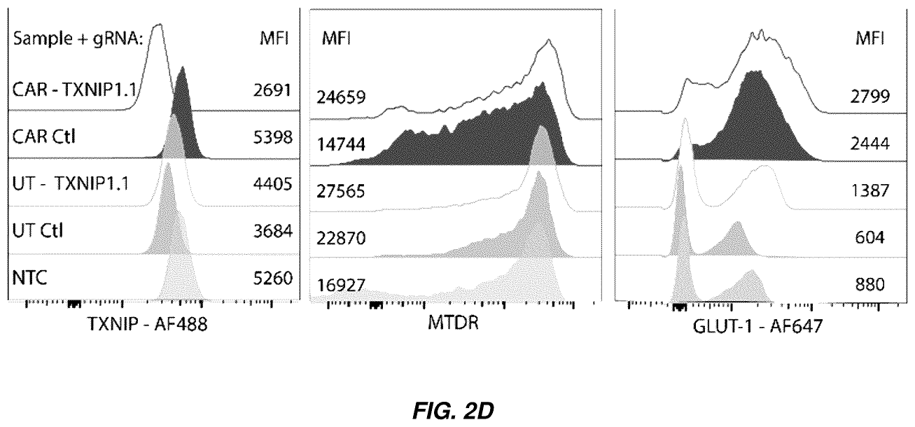

Prompted by this apparent associated between TXNIP expression and unfavorable phenotypic traits, we designed tools to either knock-out or knock-down the expression of TXNIP. FIG. 2 shows the validation of guide RNA molecules used for CRISPR/Cas9-mediated genomic ablation of the TXNIP gene. Panel A shows a schematic representation of the TXNIP gene and the location of the guide RNA targets. Panel B shows evidence of genomic editing of the TXNIP gene through a T7E1 assay, where the presence of PCR products at 400-500 bp after digestion with a mismatch-sensitive enzyme is indicative of the introduction of mutations due to non-homologous end joining. This is the result of the action of the Cas9 nuclease on the gene, which happens in presence of the targeting guide RNAs, but not in presence of a non-targeting control (NTC) or in cells that were not treated. Further validation is shown in panel C, where a reduction of the TXNIP protein is evident by Western blotting. TXNIP ablation is associated with increased GLUT1 expression and increased mitochondrial mass (Panel D).

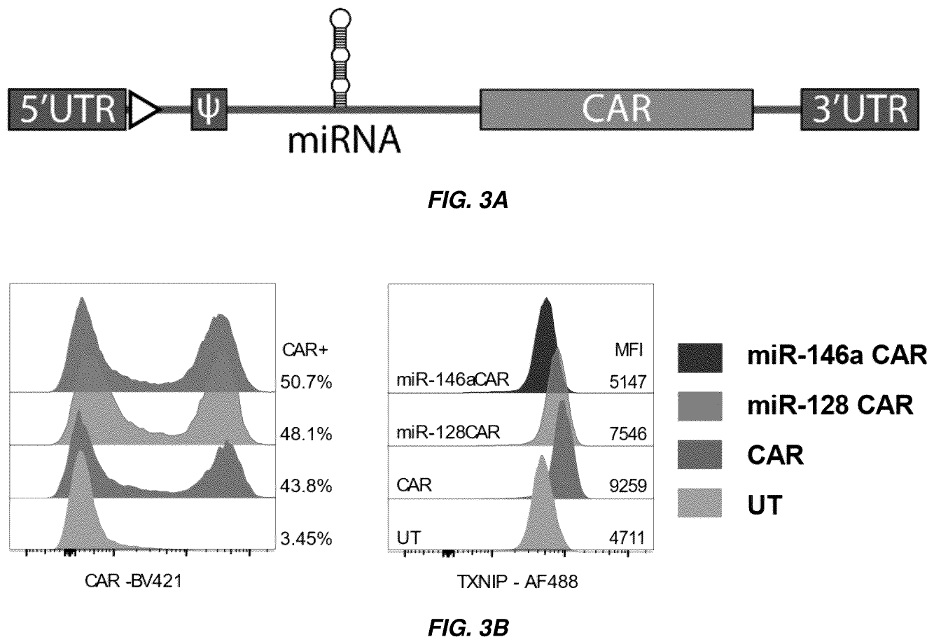

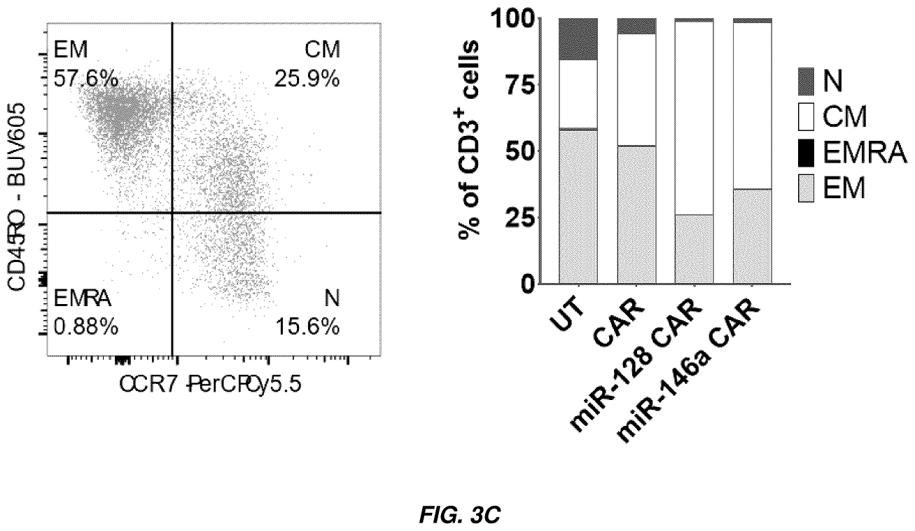

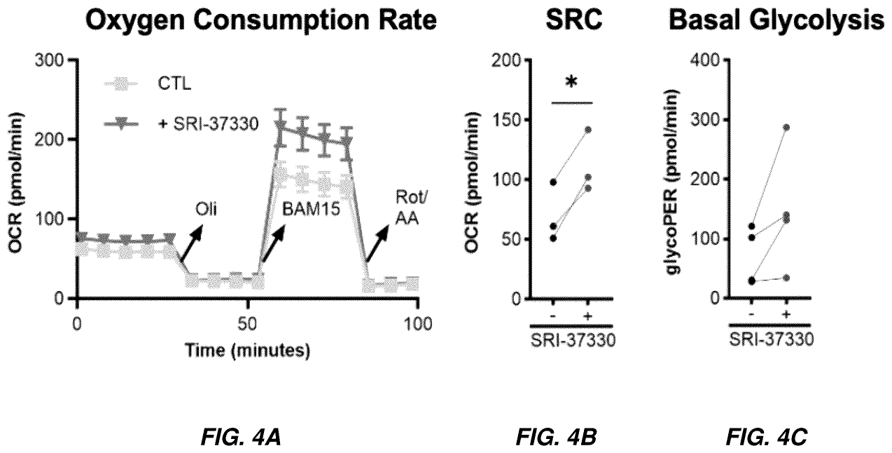

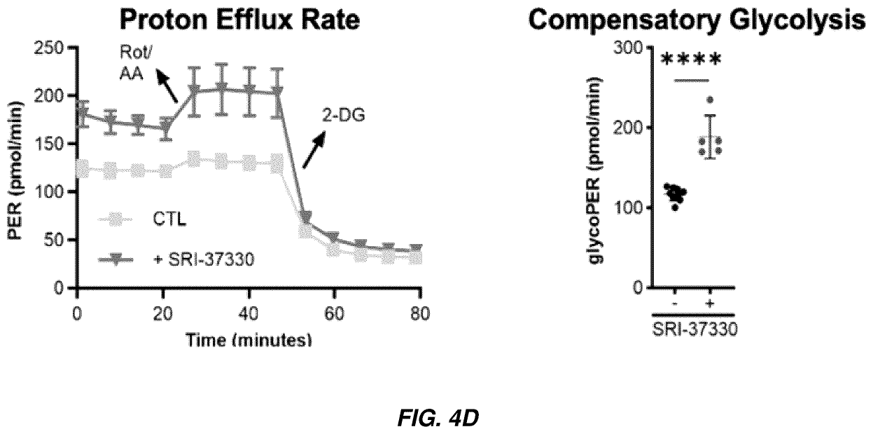

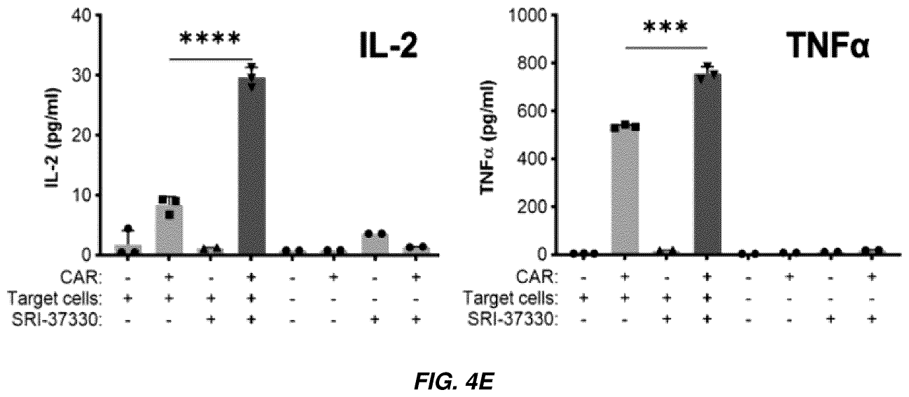

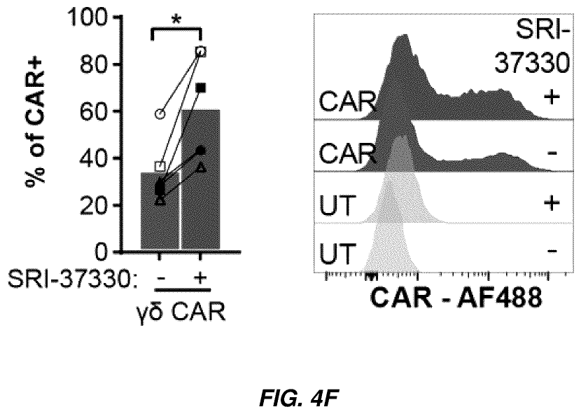

As an alternative approach, we also designed a genomic knock-down strategy based on micro-RNAs (miRNA). In this strategy, we include in the retroviral vector that induces expression of an immune receptor (for instance, a CAR), the DNA sequence that will generate a hairpin structure, which will then be processed as a microRNA. The precursor molecule is inserted in an artificial intron located upstream of the CAR cDNA, near virus encapsidation signal (FIG. 3A). As a results, upon transduction of lymphocytes, the mRNA encoding for the CAR and the intron containing the miRNA will be processed separately, leading to CAR expression and TXNIP silencing, respectively. As an example, vectors that co-express an anti-PSCA CAR together with either miRNA-128 or miRNA-146a induce similar expression of the CAR in open-repertoire T cells as the parental construct that does not express a miRNA. However, the expression of TXNIP is reduced in the T cells transduced with the miRNA vectors (FIG. 3B). This reduction of TXNIP was associated with an increase in the percentage of central memory (CM) cells, and a decrease in the percentage of effector-memory (EM) cells.

The sequence of the TXNIP exonic regions was used to identify target sequences of a panel of naturally-occurring miRNAs targeting TXNIP in other tissues, which can be have incorporated in CAR-expressing vectors. In addition, we have generated synthetic versions of the miRNAs where the hairpin sequences have been modified to achieve perfect hybridization of the TNXIP mRNA, resulting in degradation of the target mRNA, to improve the efficiency of the gene silencing. Another intended result of these synthetic modifications is to increase the specificity of the gene targeting. The target sequences of the synthetic constructs are also indicated in the figure. A list of miRNAs are provided in Table 2.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A recombinant polynucleotide comprising a
(a) a first nucleic acid sequence encoding a chimeric antigen receptor (CAR) polypeptide and/or T cell receptor; and
(b) a second nucleic acid comprising an miRNA that inhibits thioredoxin (TRX)-interacting protein (TXNIP).

2-5. (canceled)

6. A system comprising a
(a) a first nucleic acid sequence encoding a chimeric antigen receptor (CAR) polypeptide and/or T cell receptor; and
(b) a second nucleic acid sequence encoding a gRNA that targets a thioredoxin (TRX)-interacting protein (TXNIP); and
(c) a third nucleic acid sequence encoding a CRISPR-associated (Cas) endonuclease.

7-11. (canceled)

12. A method for enhancing adoptively transferred immune effector T-cells in a subject, comprising co-administering to the subject a thioredoxin (TRX)-interacting protein (TXNIP) inhibitor.

13. The method of claim 12, wherein the immune effector T-cells are γδ-T cells or T cells engineered to express a γδ-T cell receptor (TCR).

14. The method of claim 13, wherein the immune effector T-cells are tumor infiltrating lymphocytes (TILs).

15. The method of claim 12, wherein the immune effector T-cells are αβ-T cells or T cells engineered to express a αβ-T cell receptor (TCR).

16. The method of claim 12, wherein the immune effector T-cells are autologous or allogeneic.

17. The method cell of claim 12, wherein the immune effector T-cells express a chimeric antigen receptor (CAR) polypeptide.

18. The method cell of claim 12, wherein the TXNIP inhibitor comprises an siRNA, antisense, or gRNA oligonucleotide.

19. The method of claim 18, wherein the TXNIP inhibitor comprises a polynucleotide comprising a nucleic acid sequence encoding a gRNA selected from the group consisting of SEQ ID NO:1-185 and a nucleic acid sequence encoding a CRISPR-associated (Cas) endonuclease.

20. The method of claim 18, wherein the TXNIP inhibitor comprises a miRNA polynucleotide comprising a nucleic acid sequence encoding selected from the group consisting of SEQ ID NO:204-221.

21. The method cell of claim 12, wherein the TXNIP inhibitor comprises a compound represented by the formula (I-1) or the formula (I-2) or an isotopic form, a stereoisomer, a tautomer, a cis-trans isomer, a pharmaceutically acceptable salt, a pharmaceutically acceptable solvate, a hydrate, a prodrug and a polymorph thereof, 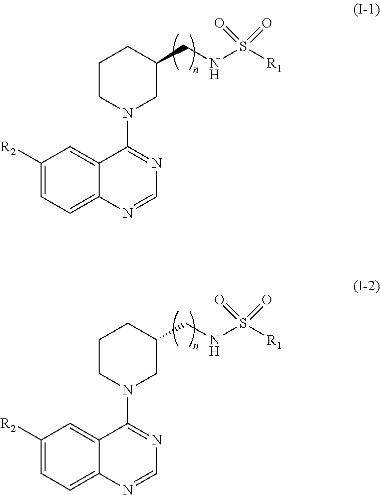
wherein, the
N is selected from any integer from 0 to 2;
R₁ is selected from one of deuterium substituted or unsubstituted C₁-C₄ alkyl, deuterium substituted or unsubstituted C₃-C₆ cycloalkyl; and
R₂ is selected from one of hydrogen, halogen, cyano, —SF₅, fluoro-substituted or unsubstituted C₁-C₄ alkyl, C₁-C₄ alkoxy, and C₃-C₆ cycloalkyl.

22. The method cell of claim 21, wherein the TXNIP inhibitor comprises a compound having the formula:

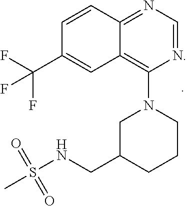 [SMILES: CS(=O)(=O)NCC1CCCN(c2ncnc3ccc(C(F)(F)F)cc23)C1]

23. The method of claim 12, further comprising administering to the subject a checkpoint inhibitor.

24. The method of claim 23, wherein the checkpoint inhibitor comprises an anti-PD-1 antibody, anti-PD-L1 antibody, anti-CTLA-4 antibody, or a combination thereof.

25. A method of providing an anti-cancer immunity in a subject, comprising
(a) administering to the subject an effective amount of a T cell expressing a gamma-delta T cell receptor (TCR) and chimeric antigen receptor (CAR) polypeptide, wherein the CAR comprises a tumor antigen binding domain, a transmembrane domain, an intracellular signaling domain, and a co-stimulatory signaling region, and
(b) administering to the subject a thioredoxin (TRX)-interacting protein (TXNIP) inhibitor.

26. The method of claim 25, wherein the TXNIP inhibitor comprises an siRNA, antisense, or gRNA oligonucleotide.

27. The method of claim 26, wherein the TXNIP inhibitor comprises a polynucleotide comprising a nucleic acid sequence encoding a gRNA selected from the group consisting of SEQ ID NO:1-185 and a nucleic acid sequence encoding a CRISPR-associated (Cas) endonuclease.

28. The method of claim 26, wherein the TXNIP inhibitor comprises a miRNA polynucleotide comprising a nucleic acid sequence encoding selected from the group consisting of SEQ ID NO:204-221.

29. The method cell of claim 25, wherein the TXNIP inhibitor comprises a compound represented by the formula (I-1) or the formula (I-2) or an isotopic form, a stereoisomer, a tautomer, a cis-trans isomer, a pharmaceutically acceptable salt, a pharmaceutically acceptable solvate, a hydrate, a prodrug and a polymorph thereof, 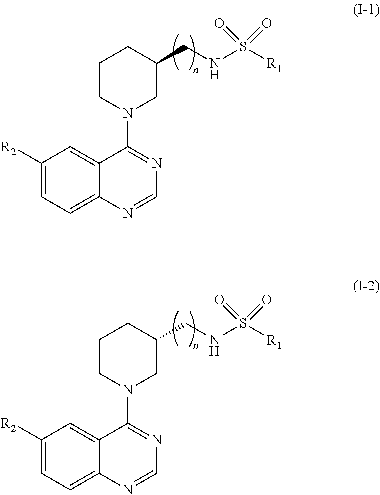
wherein, the
N is selected from any integer from 0 to 2;
R₁ is selected from one of deuterium substituted or unsubstituted C₁-C₄ alkyl, deuterium substituted or unsubstituted C₃-C₆ cycloalkyl; and
R₂ is selected from one of hydrogen, halogen, cyano, —SF₅, fluoro-substituted or unsubstituted C₁-C₄ alkyl, C₁-C₄ alkoxy, and C₃-C₆ cycloalkyl.

30. The method cell of claim 26, wherein the TXNIP inhibitor comprises a compound having the formula:

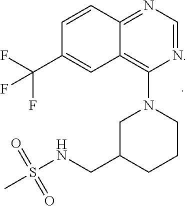 [SMILES: CS(=O)(=O)NCC1CCCN(c2ncnc3ccc(C(F)(F)F)cc23)C1]

31. The method of claim 25, further comprising administering to the subject a checkpoint inhibitor.

32. The method of claim 31, wherein the checkpoint inhibitor comprises an anti-PD-1 antibody, anti-PD-L1 antibody, anti-CTLA-4 antibody, or a combination thereof.
